(19) United States
(12) Patent Application Publication
(10) Pub. No.: US 2012/0100073 A1
(43) Pub. Date: Apr. 26, 2012

(54) PET RADIOTRACERS FOR IMAGING FATTY ACID METABOLISM AND STORAGE

(75) Inventors: Robert H. Mach, Eureka, MO (US); Robert John Gropler, St. Louis, MO (US); Zhude Tu, Frontenac, MO (US); Pilar Herrero, St.. Charles, MO (US)

(73) Assignee: WASHINGTON UNIVERSITY, St. Louis, MO (US)

(21) Appl. No.: 13/319,001

(22) PCT Filed: May 4, 2010
(86) PCT No.: PCT/US10/33579

§ 371 Date: Dec. 28, 2011

Related U.S. Application Data
(60) Provisional application No. 61/175,065, filed on May 4, 2009.

(51) Int. Cl. A61K 51/04; C07C 67/08; C07C 69/734; C07C 51/09; C07C 59/64; C07D 249/04

(52) U.S. Cl. 424/1.89; 554/218; 548/255; 554/163; 554/165

(57) ABSTRACT

Fatty acid analogue (FAA) molecules comprising positron-emitting radionuclides, salts thereof, and FAA-triglycerides are disclosed. Also disclosed are methods of synthesis, and methods of imaging distribution and metabolism of fatty acids and fatty acid triglycerides.

### PRIORITY STATEMENT

This application claims the benefit of and the priority to U.S. Provisional Application No. 61/175,065, filed on May 4, 2009, which is incorporated herein by reference in its entirety.

### GOVERNMENT SUPPORT

This work was supported at least in part by NIH grant HL69100. The government may have certain rights in the invention.

### INTRODUCTION

The present teachings are in the field of tracers that can be used for imaging distribution and metabolism of fatty acids and fatty acid triglycerides.

Distribution of fatty acids, including complexes of fatty acids FA) with triglycerides, (FA-TG) is of great clinical significance in various tissues such as cardiac tissue.

Many probes and methods have been developed for imaging distribution of Fatty acids in subjects such as humans.

³¹P and ¹³C magnetic resonance spectroscopy (MRS) have been used to image myocardial substrate metabolism in ex-vivo preparations in vivo.^{1,5-7} However, because of the inherent low signal-to-noise of the magnetic resonance method, limited spatial resolution, intravoxel signal contamination and long acquisition times, assessment of myocardial metabolism in vivo is limited only to the anterior myocardium.

Radiolabeled 15-(p-iodophenyl)-pentadecanoic acid (IPPA) has been used as a radiotracer for imaging FA metabolism using single photon emission computed tomography (SPECT).⁸⁻¹¹ However, SPECT systems do not have the temporal resolution to take advantage of the rapid turnover of IPPA to permit high quality imaging and quantification of FA metabolism.

Branched-chain analogs of IPPA, such as BMIPP, have also been developed as tracers for FA metabolism.^{10,2-14} However, quantification of myocardial substrate use is difficult or impossible because SPECT provides relatively poor temporal and spatial resolution and inaccurate correction for photon attenuation, and incomplete metabolism of BMIPP relative to unlabeled FA use.

¹¹C-palmitate has been used as a radiotracer for PET imaging of FA metabolism in the heart.² However, image quality is generally considered low. In addition, radiolableled metabolite corrections are frequently needed. Finally, the short half-life of the carbon-11 radioisotope (˜20 min) necessitates rapid access to sources such as a cyclotron and a radiopharmaceutical production apparatus.

14-(R,S)-¹⁸F-fluoro-6-thiaheptadecanoic acid (FTHA) has been used as a radiotracer for PET imaging of FA metabolism.^{15,16} However, uptake and retention of SF-FT-IA is insensitive to the inhibition of β-oxidation by hypoxia.¹⁷

¹⁸F-fluoro-thia-palmitate (FTP) is a probe for PET imaging of a metabolic trapping function.¹⁷ Deposition of FTP is proportional to β-oxidation under normal oxygenation and hypoxic conditions. However, FTP does not distinguish between myocardial FA uptake and oxidation.¹⁸ Moreover, quantification of these processes requires correction for the intrinsic differences in the kinetics of FTP and unlabeled FA¹⁸.

Trans-9(RS)-[F-18]-fluoro-3,4(RS,RS) methyethyienebeptadecanoic acid (¹⁸F-FCPHA), has been described with a structure that includes a cyclopropyl group at C3-C4 and an alkyl fluoride at C9.¹⁹ However, the impact of alterations in plasma substrates, work load and blood flow on myocardial kinetics is unknown.

A need therefore exists for radiotracers that provide accurate and comprehensive measurements of myocardial FA metabolism.

### SUMMARY

The present inventors have developed fatty acid analog (FAA) compounds and salts thereof having structures in various aspects of the present teachings, such as

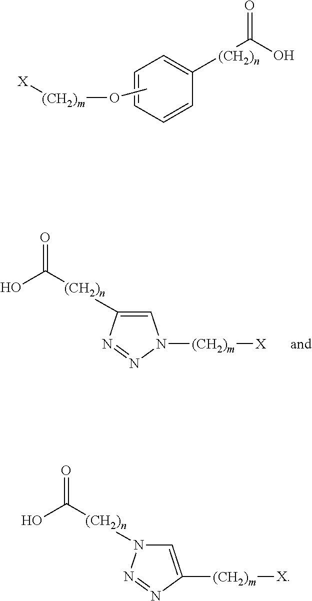

In various configurations, n can be an integer from 10 to 24, i can be an integer from 1 to 10, and X can be a halogen. In some configurations, a fatty acid analog or salt thereof of the present teachings can have structure

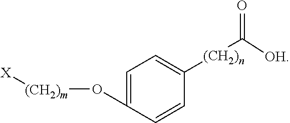

A fatty acid analog or salt thereof of the present teachings can include at least one radioisotope. In some configurations, a radioisotope can be a positron-emitting radioisotope. In some con-figurations, n can be 14; independently, m can be 2, and independently, X can be a fluorine atom such as a ¹⁸F radioisotope. In some configurations, n=14, m=2 and X is ¹⁸F.

In various aspects, a compound or salt thereof of the present teachings can be used as a probe for fatty acid distribution in a subject such as a mammal, including a human mammal. Following administration to a subject mammal such as a human, distribution of a radiotracer of the present teachings can be determined by methods known to skilled artisans, such as positron emission tomography (PET) scanning or single photon emission computed tomography. In certain configurations, a fatty acid analog or salt thereof of the present teachings can provide myocardial kinetics that closely mimic those of ¹¹C-palmitate.

In various aspects, the present teachings also include fatty acid analog-triglycerides (FAA-TG) and salts thereof. In some configurations, an FAA-TG can be fatty acid analog-very low density lipoprotein triglyceride (FAA-VLDL). In some configurations, an FAA-TG or FAA-VLDL can comprise a radioisotope such as an ¹⁸F. Accordingly, the present teachings include in some configurations various ¹⁸F-fatty acid analog-very low density lipoprotein triglycerides such as ¹⁸F-FAA-TG's and ¹⁸F-FAA-VLDL's. In some embodiments, an ¹⁸F-FAA-TG or ¹⁸F-FAA-VLDL or a salt thereof of the present teachings can have a structure

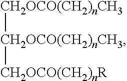

wherein R can be

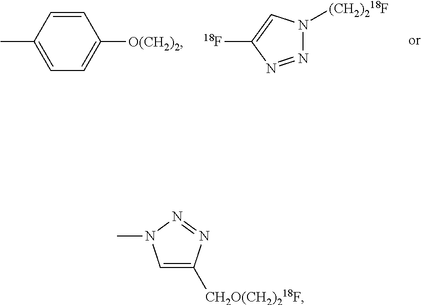

wherein n is an integer from 10 to 24. In some configurations, n can be 14.

In various aspects of the present teachings, the present inventors disclose methods of synthesizing the fatty acid analogs, as well as various intermediates that are useful for synthesizing fatty acid analogs.

In some aspects, the inventors disclose methods of synthesizing Br—(CH₂)ₙ—COOCtF₃ 2. These methods can comprise contacting Br—(CH₂)ₙ—COOH 1 with trimethylsilyl diazomethane, THF, hexane, wherein n is an integer from 10 to 24. In some configurations, n can be 14.

In some aspects, the inventors disclose methods of synthesizing

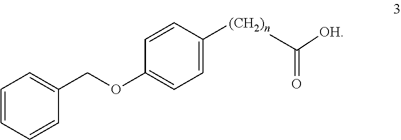

These methods can comprise contacting Br—(CH₂)ₙ—COOCH₃ 2 with 4-benzyloxyphenylboronic acid, Pd(OAc), [HP(t-Bu)₂Me]BF₄, KOt-Bu and t-amyl alcohol, wherein a can be an integer from 10 to 24. In some configurations, n can be 14.

In some aspects, the inventors disclose methods of synthesizing

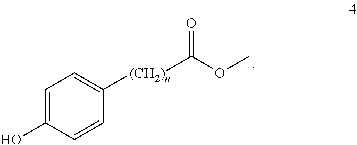

These methods can comprise contacting

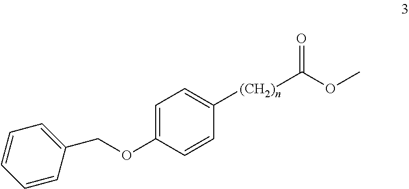

with Pd/c, EtOAc, H₂, wherein n can be an integer from 10 to 24. In some configurations, n can be 14.

In some aspects, the inventors disclose methods of synthesizing

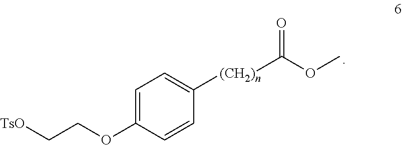

These methods can comprise contacting

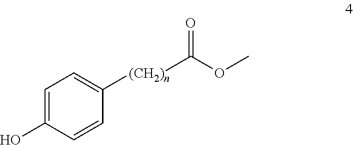

with ethane-1-2-diyl bis(4-methylbenzenesulfonate) K₂CO₃, CH₃CN, wherein n can be an integer from 10 to 24. In some configurations, n can be 14.

In some aspects, the inventors disclose methods of synthesizing

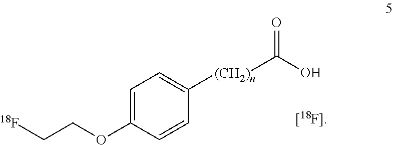

These methods can comprise contacting

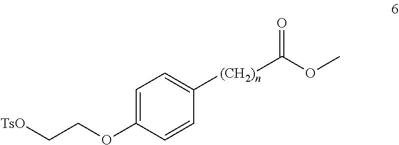

with [¹⁸F]KFK2.2.2/K₂CO₃/CH₃CN, then NaOH,
wherein n can be an integer from 10 to 24. In some configurations, n can be 14. K2.2.2 is 4,7,13,16,21,24-Hexaoxa-1,10-diazabicyclo[8.8.8]-hexacosane (Kryptofix 222®, Acros Organics N.V., Fairlawn N.J.).

In some configurations, these methods can further comprise a) contacting Br—(CH₂)ₙ—COOH 1 with trimethylsilyl diazomethane and THF to yield Br—(CH₂)ₙ—COOCH₃ 2; b) contacting the Br—(CH₂)ₙ—COOCH₃ 2 with 4-benzyloxyphenylboronic acid, Pd(OAc)₂, [Hp(t-Bu)₂Me]BF₄, KOt-Bu and t-amyl alcohol to obtain

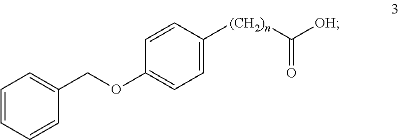

c) contacting the

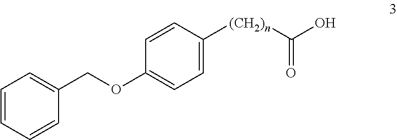

with of Pdc and EtOAc to obtain

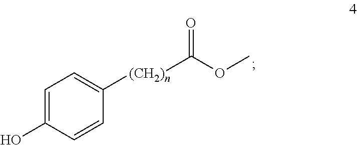

and d) contacting the

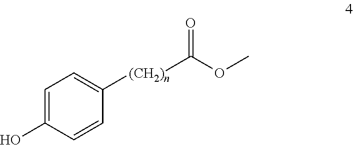

with ethane-1-2-diyl bis(4-methylbenzenesulfonate), K₂CO₃ and CH₃CN to obtain

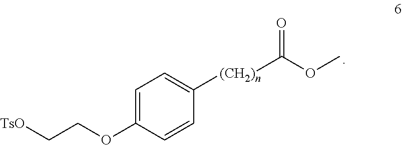

In some aspects, the inventors disclose methods of synthesizing

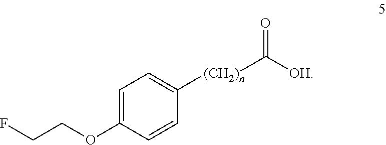

In various configurations, these methods can comprise contacting

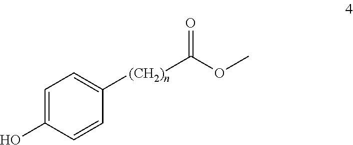

with 1-bromo-2-fluoroethane, K₂CO₃, acetone; followed by NaOH, MeOH, CHCl₂, water, wherein n can be an integer from 10 to 24, and m is an integer from 1 to 10. In some configurations, n can be 14.

In some configurations, these methods can further comprise a) contacting Br—(CH₂)ₙ—COOH 1 with trimethylsilyl diazomethane and THF to yield Br—(CH₂)ₙ—COOCH₃ 2; b) contacting the Br—(CH₂)ₙ—COOCH₃ 2 with 4-benzyloxyphenylboronic acid, Pd(OAc)₂, [Hp(t-Bu)₂Me]BF₄, KOt-Bu and t-amyl alcohol to obtain.

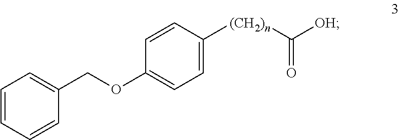

c) contacting the

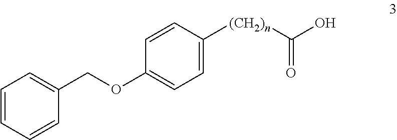

with of Pd/c and EtOAc to obtain

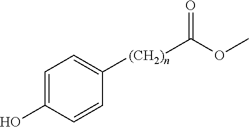

In some configurations, the present teachings include methods of synthesizing.

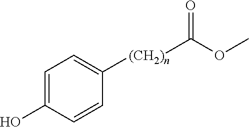

In various configurations, these methods can include contacting Br—(CH₂)₁₄—COOCH 1 with trimethylsilyl diazomethane and THF to obtain Br—(CH₂)ₙ—COOCH₃ 2; contacting the Br—(CH₂)ₙ—COOCH₂ with 4-benzyloxyphenylboronic acid, Pd(OAc)₂, [HP(t-Bu₂-Me]BF₄, KOt-Bu and t-amyl alcohol to obtain

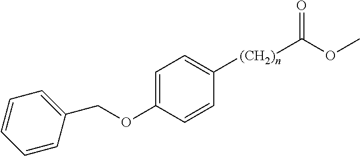

and contacting the

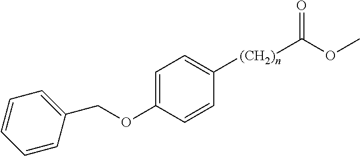

with Pd/c, EtOAc, H₂ to yield

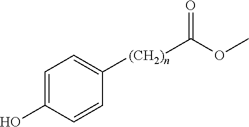

wherein n can be an integer from 10 to 24. In some configurations, n can be 14.

In some aspects, the present inventors disclose methods of synthesizing

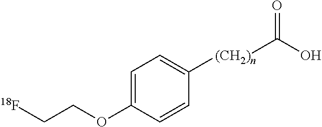

In various configurations, these methods can comprise contacting

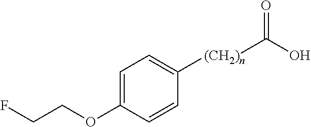

with [¹⁸F]KF/K2.2.2/K₂CO₂/CH₃CN, then NaOH
wherein n is an integer from 10 to 24. In some configurations, n can be 14. In addition, in some configurations, these methods can further comprise contacting Br—(CH₂)₁₄—COOH 1 with trimethylsilyl diazomethane and THF to obtain Br—(CH₂)ₙ—COOCH₃ 2; contacting the Br—(CH₂)ₙ—COOCH₃ 2 with 4-benzyloxyphenylboronic acid, Pd(OAc)₂, [HP(t-Bu)₂Me]BF₄, KOt-Bu and t-amyl alcohol to obtain

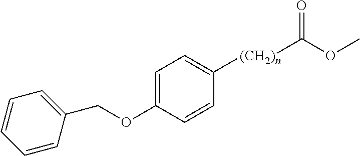

contacting the

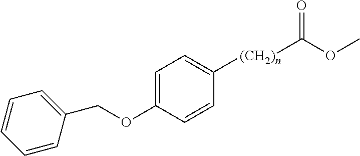

with Pd/c, EtOAc, H₂ to obtain

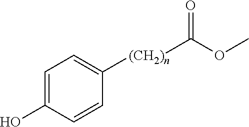

and contacting the

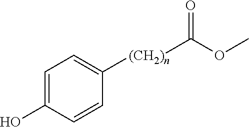

with 1-bromo-2-fluoroethane, K₂CO₃, acetone; followed by NaOH, MeOH, CHCl₂, water, to obtain

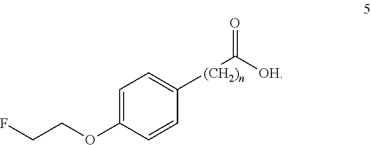

In various aspects, the present teachings include methods of synthesizing

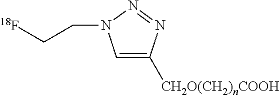

In various configurations, these methods can include contacting Br—(CH₂)ₙ—COOH 1 with

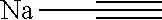

to obtain

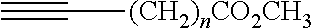

7; and contacting the

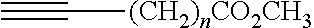

7 with

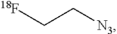

wherein n is an integer from 10 to 24. In some configurations, n can be 14.

In various aspects, the present teachings include methods of synthesizing

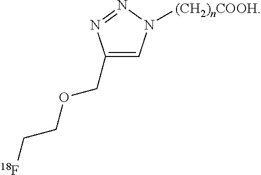

In various configurations, these methods can include contacting Br—(CH₂)ₙ—COOH 1 with NaN₃ to form N₃(CH₂)ₙCOOCH₃ 8; and can be an integer from 10 to 24. In some configurations, n can be 14.

In various aspects, the present teachings include methods of synthesizing

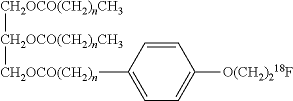

1,2-Pal-[¹⁸F]5. In various configurations, these methods can include contacting

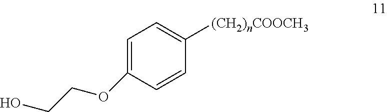

with TMS—Cl and CH₂Cl₂ to form

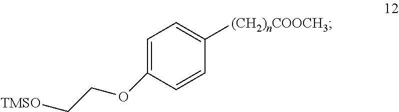

contacting the

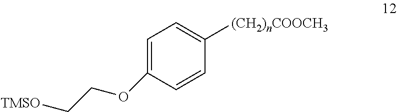

with NaOH and ethanol to form

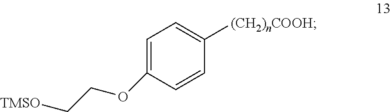

contacting the

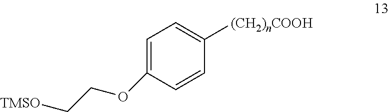

with

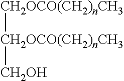

and DCC/CH₂Cl₂ to form

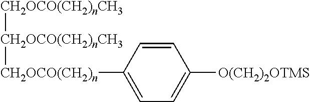

and contacting the

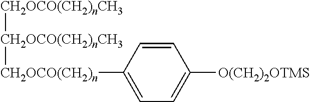

with TBAF, then Tf₂O/CH₂Cl₂, then labeling, wherein n can be an integer from 10 to 24. In some configurations, n can be 14.

In various aspects, the present teachings include methods of synthesizing

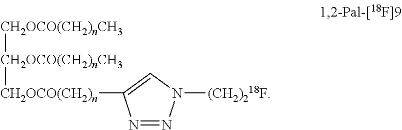

In various configurations, these methods can include contacting

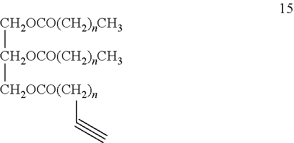

with

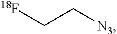

wherein n is an integer from 10 to 24. In some configurations, n can be 14.

In various aspects the present teachings include methods of synthesizing

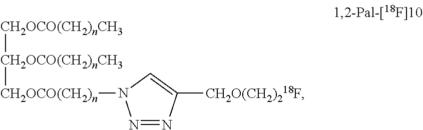

In various configurations these methods ca include contacting

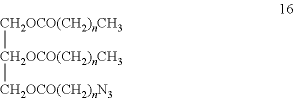

with

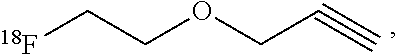

wherein n is an integer from 10 to 24. In some configurations, n can be 14.

In some aspects of the present teachings, “click” analogs can be prepared as outlined in FIG. 1. In some configurations, a 4-iodophenyl ring of IPPA can be replaced with a corresponding 1,2,3-triazole moiety that is created from the “click” or “reverse click” labeling procedures. The synthesis of the target compounds and precursors for labeling are disclosed herein.

In some aspects of the present teachings, the inventors disclose methods of determining fatty acid distribution in an mammal, such as a laboratory animal, a companion animal, a livestock animal, or a human. In various configurations, these methods can comprise: administering to a mammal a radiolabeled fatty acid analog or salt thereof selected such as

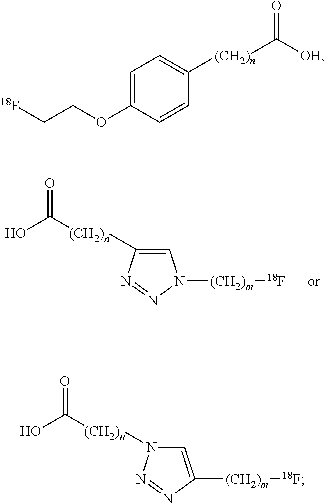

and subjecting the mammal to positron emission tomography (PET) or other positron detection methods known to skilled artisans such as SPECT. In some configurations, m can be 2. In some configurations, n can be 14. In various configurations, imaging data obtained from a scan can be record on a digital computer and analyzed using methods known to skilled artisans, such as analysis using known algorithms. In some configurations, analysis using an algorithm can be accomplished with the aid of a digital computer. In some embodiments, the methods can further include displaying the fatty acid distribution in a subject mammal as an image on a computer display.

In some aspects, the present teachings include methods of imaging distribution of fatty acid triglycerides in a mammal. In various configurations, the methods can include administering to a mammal a radiolabeled fatty acid triglyceride analog or salt thereof, such

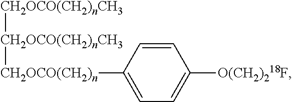

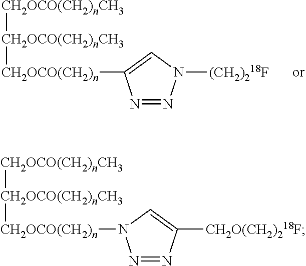

and subjecting the mammal to positron emission tomography (PET) scanning or other positron imaging methods such as SPECT, wherein n can be an integer from 10 to 24. In some configurations, n can be 14. In some configurations, imaging data resulting from a scan can be stored on a digital computer. In some embodiments, these methods can further comprise subjecting image data to analysis using an algorithm known to skilled artisans. In some configurations, an algorithm can be stored on a digital computer. In some configurations, the methods can further comprise displaying fatty acid triglyceride distribution in a subject mammal on a computer display.

### Aspects

The present inventors' disclosure includes the following aspects.

1. A fatty acid analog or salt thereof of structure

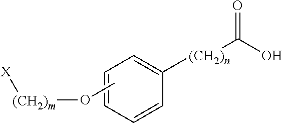

wherein n is an integer from 10 to 24, m is an integer from 1 to 10, and X is a halogen.
2. A fatty acid analog or salt thereof in accordance with aspect 1, wherein the fatty acid analog is

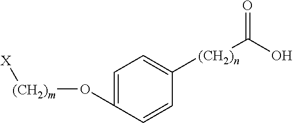

3. A fatty acid analog or salt thereof in accordance with aspect 1 or aspect 2, wherein X is a fluorine.
4. A fatty acid analog or salt thereof in accordance with any one of aspects 1-3, wherein at least one atom is a radioisotope.
5. A fatty acid analog or salt thereof in accordance with any one of aspects 1-3, wherein at least one atom is a positron-emitting radioisotope.
6. A fatty acid analog or salt thereof in accordance with any one of aspects 1-3 wherein X is an ¹⁸F.
7. A fatty acid analog or salt thereof in accordance with any one of aspects 1-6, wherein m=2.
8. A fatty acid analog or salt thereof in accordance with any one of aspects 1-7, wherein n=14.
9. A fatty acid analog or salt thereof in accordance with aspect 1, wherein n=14, m=2 and X is ¹⁸F.
10. A fatty acid analog or salt thereof of structure selected from the group consisting of

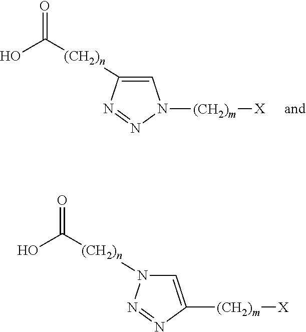

wherein n is an integer from 10 to 24, m is an, integer from 1 to 10, and X is a halogen.
11. A fatty acid analog or salt thereof in accordance with aspect 10, of structure

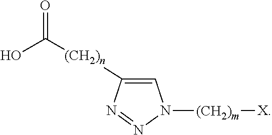

12. A fatty acid analog or salt thereof in accordance with aspect 10, of structure

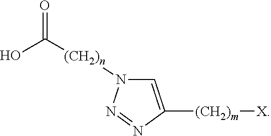

13. A fatty acid analog or salt thereof in accordance with any one of aspects 10-12, wherein X is a fluorine atom.
14. A fatty acid analog or salt thereof in accordance with any one of aspects 10-13, wherein at least one atom is a radioisotope.
15. A fatty acid analog or salt thereof in accordance with any one of aspects 10-13, wherein at least one atom is a positron-emitting radionuclide.
16. A fatty acid analog or salt thereof in accordance with any one of aspects 10-15, wherein X is an ¹⁸F.
17. A fatty acid analog or salt thereof in accordance with any one of aspects 10-16, wherein m=2.
18. A fatty acid analog or salt thereof in accordance with any one of aspects 10-17, wherein n=14.
19. A fatty acid analog or salt thereof in accordance with aspect 11, of structure

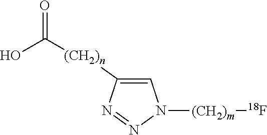

wherein n=14 and m=2.
20. A fatty acid analog or salt thereof in accordance with aspect 12, of structure

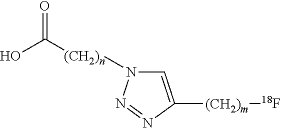

wherein n=14 and m=12.
21. An ¹⁸F-fatty acid analog-very low density lipoprotein triglyceride (¹⁸F-FAA-VLDL) of structure

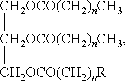

wherein R is selected from the group consisting of

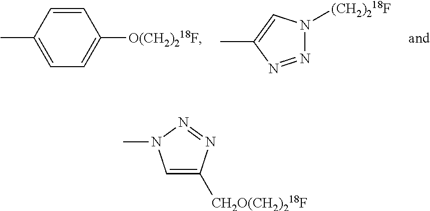

or a salt thereof, wherein n is an integer from 10 to 24.
22. An ¹⁸F-FAA-VLDL or salt thereof in accordance with aspect 21, wherein n=14.
23. A method of synthesizing Br—(CH₂)ₙ—COOCH₃ 2, comprising contacting Br—(CH)ₙ—COOH 1 with trimethylsilyl diazomethane, THF, hexane, wherein n is an integer from 10 to 24.
24, A method of synthesizing Br—(CH₂)ₙ—COOCH₃ 2 in accordance with aspect 23, wherein n=14.
25. A method of synthesizing

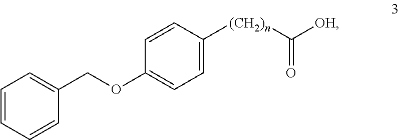

comprising contacting Br—(CH₂)ₙ—COOCH₃ 2 with 4-benzyloxyphenylboronic acid, Pd(OAc)₂, [HP(t-Bu)₂Me]BF₄, KOt-Bu and t-amyl alcohol, wherein n is an integer from 10 to 24.
26. A method in accordance with aspect 25, wherein n=14.
27. A method of synthesizing

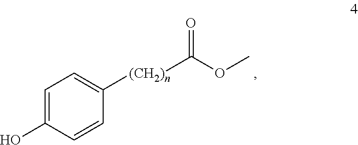

comprising contacting

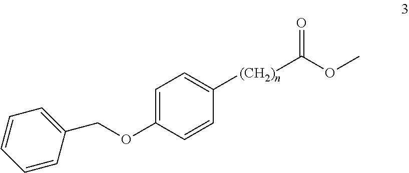

with Pd/c, EtOAc, H₂, wherein n is an integer from 10 to 24,
28. A method in accordance with aspect 27, wherein n=4.
29. A method of synthesizing

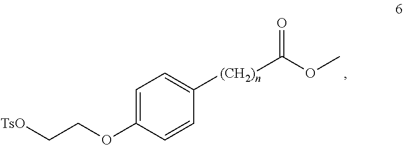

comprising contacting

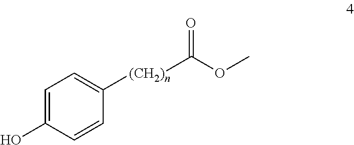

with ethane-1-2-diyl bis(4-methylbenzenesulfonate), K₂CO₃, CH₃CN, wherein n is an integer from 10 to 24.
30. A method in accordance with aspect 29, wherein n=14.
31. A method of synthesizing

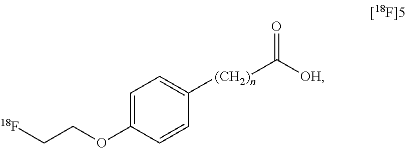

comprising contacting

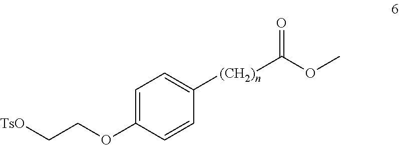

with [¹⁸F]KF/K2.2.2/K₂CO₃/CH₃CN, then NaOH,
wherein n is an integer from 10 to 24.
32. A method in accordance with aspect 31, wherein n=14.
33. A method in accordance with aspect 31 or aspect 32, further comprising:

a) contacting Br—(CH₂)—COOH 1 with trimethylsilyl diazomethane and THF to yield Br—(CH₂)ₙ—COOCH₃ 2;

b) contacting the Br—(CH₂)ₙ—COOCH₃ 2 with 4-benzyloxyphenylboronic acid, Pd(OAc)₂, [H-p(t-Bu)₂Me]BF₄, KOt-Bu and t-amyl alcohol to yield

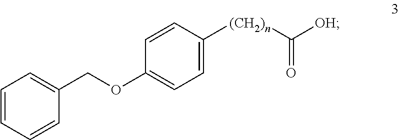

c) contacting the

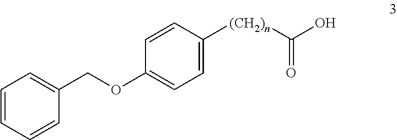

with of Pd/c and EtOAc to yield

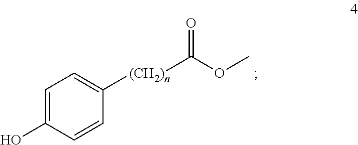

d) contacting the

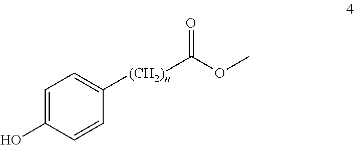

with ethane-1-2-diyl bis(4-methylbenzenesulfonate), K₂CO₃ and CH₃CN to yield

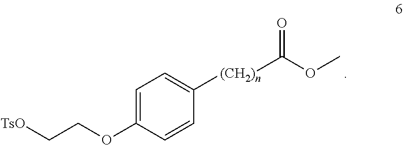

34. A method of synthesizing

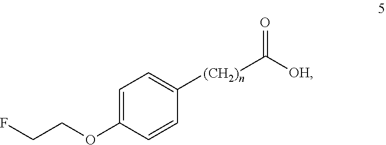

comprising contacting

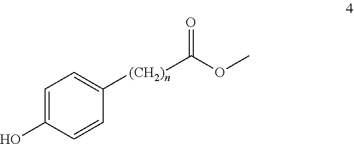

with 1-bromo-2-fluoroethane, K₂CO₃, acetone; followed by NaOH, MeOH, CHCl₂, water,
wherein n is an integer from 10 to 24, and m is an integer from 1 to 10.
35. A method in accordance with aspect 34, wherein n=14.
36. A method in accordance with aspect 34 or 35, further comprising
a) contacting Br—(CH₂)ₙ—COOH 1 with trimethylsilyl diazomethane and THF to yield Br—(CH₂)ₙ—COOCH₃ 2;
b) contacting the Br—(CH₂)ₙ—COOCH₃ 2 with 4-benzyloxyphenylboronic acid, Pd(OAc)₂, [Hp(t-Bu)₂Me]BF₄, KOt-Bu and t-amyl alcohol to yield

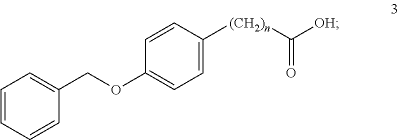

c) contacting the

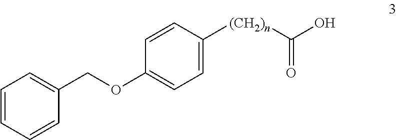

with of Pdic and EtOAc to yield

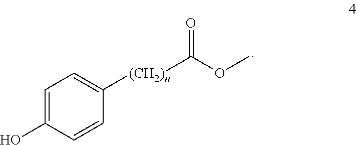

37. A method of synthesizing

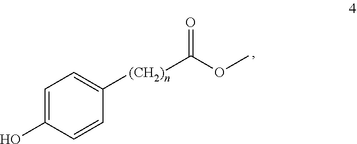

comprising:

contacting Br—(CH₂)ₙ—COOH 1 with trimethylsilyl diazomethane and THF to yield Br—(CH₂)ₙ—COOCH₃ 2:

contacting the Br—(CH₂)ₙ—COOCH₃ 2 with 4-benzyloxyphenylboronic acid, Pd(OAc)₂, [HP(t-Bu)₂Me]BF₄, KOt-Bu and t-amyl alcohol to yield

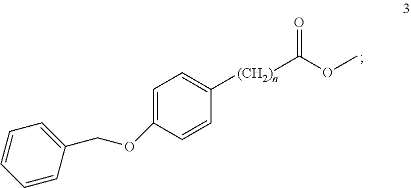

and

contacting the

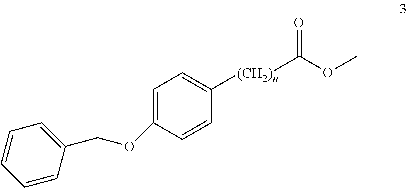

with Pd/c, EtOAc, H₂ to yield

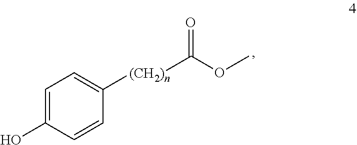

wherein n is an integer from 10 to 24.
38. The method of aspect 37, wherein n=14.
39. A method of synthesizing

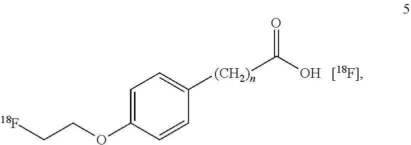

comprising:
contacting

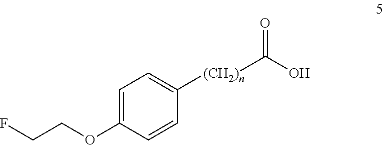

with [¹⁸F]KF/K2.2.2/K₂CO₃/CH₃CN, then NaOH
wherein n is an integer from 10 to 24.
40. The method of aspect 39, wherein n=14.
41. The method of aspect 39 or 40, further comprising

contacting Br—(CH₂)₁₄—COOH 1 with trimethyllsilyl diazomethane and THF to yield Br—(CH₂)ₙ—COOCH₃ 2;

contacting the Br—(CH₂)ₙ—COOCH₃ 2 with 4-benzyloxyphenylboronic acid, Pd(OAc)₂, [HP(t-Bu)₂Me]BF₄, KOt-Bu and t-amyl alcohol to yield

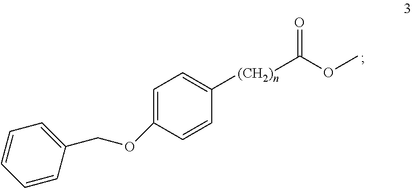

and contacting the

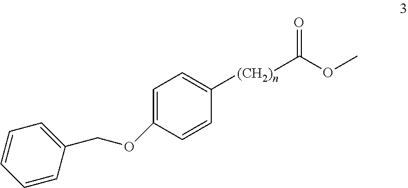

with Pd/c, EtOAc, H₂ to yield

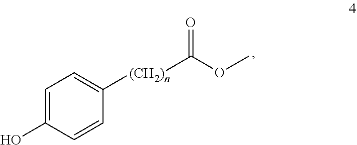

contacting the

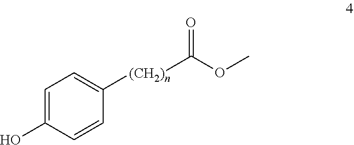

with 1-bromo-2-fluoroethane, K₂CO₃, acetone; followed by NaOH, MeOH, CHCl₂, water, to yield

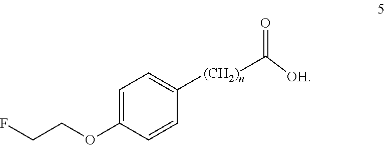

42. A method of synthesizing

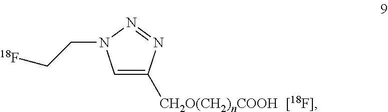

comprising:

contacting Br—(CH₂)ₙ—COOH 1 with

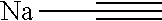

to form

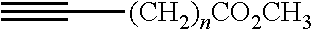

7;
contacting the

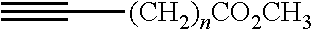

with

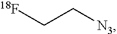

wherein n is an integer from 10 to 24.
43. A method in accordance with aspect 42, wherein n=14.
44, A method of synthesizing

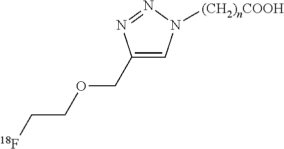

comprising:

contacting Br—(CH₂)ₙ—COOH 1 with NaN₃ to form N₃(CH₂)ₙCOOCH₃ 8; and

contacting the N₃(CH₂)ₙCOOCH₃ 8 with

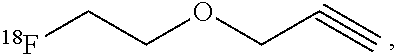

wherein n is an integer from 10 to 24.
45. A method in accordance with aspect 44, wherein n=14.
46. A method of synthesizing

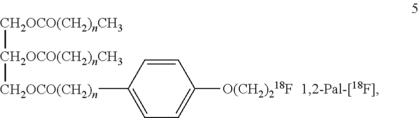

comprising:

contacting

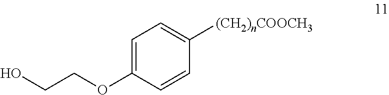

with TMS-Cl and CH₂Cl₂ to form

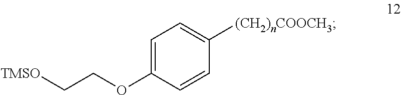

contacting the

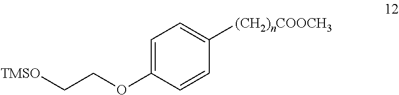

with NaOH and ethanol to form

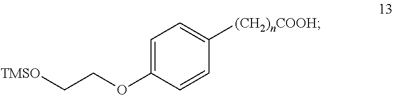

contacting the

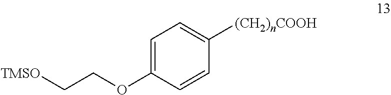

with

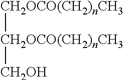

and DCC/CH₂Cl₂ to form

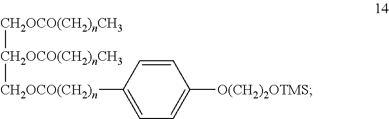

and

contacting the

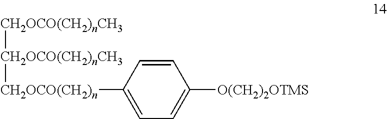

with TBAF, then Tf₂O/CH₂Cl₂, then labeling, wherein n is an integer from 10 to 24.
47. A method in accordance with aspect 46, wherein n=14.
48. A method of synthesizing

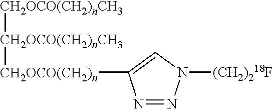

1,2-Pal-[₁₈F]9, comprising:

contacting

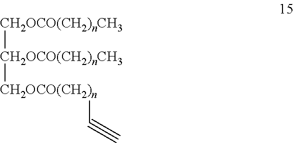

with

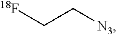

wherein n is an integer from 10 to 24.

49. A method in accordance with aspect 48, wherein n=14.

50. A method of synthesizing

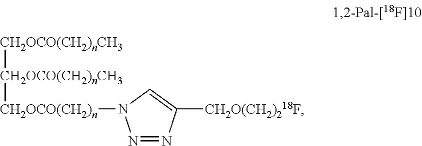

comprising contacting

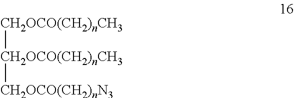

with

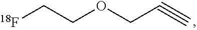

wherein n is an integer from 10 to 24.
51. A method in accordance with aspect 50, wherein n=14.
52. A method of determining fatty acid distribution in an mammal, comprising: administering to the mammal a radiolabeled fatty acid analog or salt thereof selected from the group consisting of

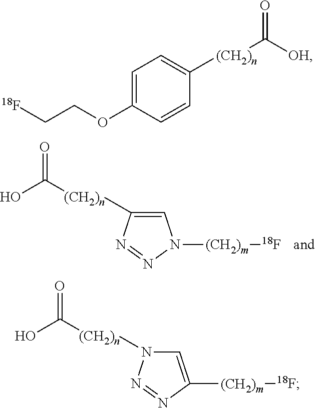

and subjecting the mammal to positron emission tomography (PET) scanning, wherein n is an integer from 10 to 24 and m is an integer from 1 to 10.
53. A method in accordance with aspect 52, wherein m=2.
54. A method in accordance with aspect 52 or aspect 53, wherein n=14.
55. A method in accordance with any one of aspects 5254, further comprising subjecting image data to analysis by an algorithm in a digital computer.
56. A method in accordance with any one of aspects 51-55, further comprising displaying the fatty acid distribution in an image on a computer display.
57. A method of imaging distribution of fatty acid triglycerides in a mammal, comprising:

administering to the mammal a radiolabeled fatty acid triglyceride analog or salt thereof selected from the group consisting of

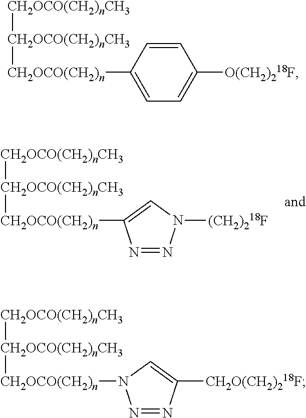

and

subjecting the mammal to positron emission tomography (PET) scanning, wherein n is an integer from 10 to 24.

58. A method in accordance aspect 57, wherein n=14.
59. A method in accordance aspect 57 or aspect 58, further comprising:

subjecting image data to analysis by an algorithm in a digital computer.

60. A method in accordance with any one of aspects 57-59, further comprising displaying fatty acid triglyceride distribution on a computer display.

### BRIEF DESCRIPTION OF THE DRAWINGS

FIG. 1 illustrates the strategy used in the design of F labeled fatty acid analogs based on p-IPPA.

FIG. 2 illustrates synthesis pathways for

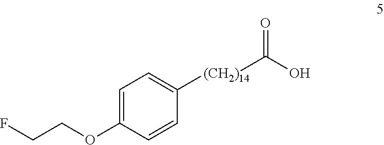

and

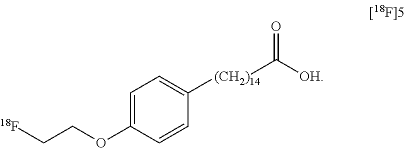

Reagents: a: trimethylsilyl diazomethane, T-F, hexane, 2 hr; b: 4-benzyl oxyphenylboronic acid, Pd(OAc)₂, [HP(t-Bu)₂Me]BF₄, KOt-Bu, t-amyl alcohol, argon, RT, 24 hr; c: Pd/c, EtOAc, H₂, 6 psi, 5 hr; d: 1-bromo-2-fluoroethane, K₂CO₃, acetone; e: NaOH, MeOH, CHCl₂, water; f: ethane-1,2-diyl bis(4-methylbenzenesulfonate), K₂CO₃, CH₃CN reflux 3 hr; g: [¹⁸F]KF/K2.2.2/K₂CO₃/CH₃CN, then NaOH.

FIG. 3 illustrates “traditional” (top) and “reverse” (bottom) click chemistry used in the preparation of some ¹⁸F-FAA compounds of the present teachings.

FIG. 4 illustrates myocardial microPET images obtained from a fed rat studied with ¹¹C-palmitate (top) and the compound [¹⁸F]5 (bottom). Images are displayed on the transaxial axis. A composite image, and images of individual RGB color channels are shown.

FIG. 5 illustrates blood (input) and myocardial time-activity curves (TACs) (anterior and lateral) for ¹¹C-palmitate (top) and the ¹⁸F-FAA (bottom).

FIG. 6 illustrates the structures of some ¹⁸F-labeled triglyceride analogs.

FIG. 7 illustrates a synthesis scheme for ¹⁸F-labeled FA analogs 12 and 13, each comprising a phenyl moiety.

FIG. 8 illustrates a synthesis scheme for ¹⁸F-labeled triglyceride analogs 14 and 15, each comprising a phenyl moiety,

FIG. 9 illustrates a synthesis scheme for ¹⁸F-labeled triglyceride analogs 1,2-Pal-[¹⁸F]9 and 1,2-Pal-[¹⁸F]10, each comprising a triazole moiety.

### DETAILED DESCRIPTION

Myocardial fatty acid (FA) oxidation is believed to be among the heart's most important energy sources. However, the proportional contribution of other substrates to overall oxidative metabolism such as glucose and lactate are both significant and quite variable and dependent upon numerous factors such as the plasma substrate environment, neurohumoral milieu and level of cardiac work. Thus, plasticity in myocardial substrate use can be key to cardiac health. Loss of plasticity resulting in near exclusive use of one substrate has been shown to have a role in the development of ventricular dysfunction in a variety of cardiac disease processes.

Myocardial FA metabolism is believed to be dependent on the plasma delivery of FA, either in the form of FA bound to albumin (FA-ALB) or in triglyceride (FA-TG) (either in the form of chlyomicrons (FA-CM) or very low density lipoproteins (FA-VLDL)) with subsequent release of the FA via lipoprotein lipase (LPL) located on capillary endothelial. Furthermore, given the potential adverse effects of either accelerated fatty acid oxidation or excessive lipid accumulation, the present inventors have seen a need for PET radiotracers that can track FA arising from FA-TG. In some embodiments, the inventors disclose ¹⁸F-FAA with kinetics similar to those of unlabeled palmitate, and furthermore disclose ¹⁸F-FAA incorporated into the 1-position of a triglyceride.

The present inventors have developed imaging approaches that, in some embodiments, further extend our capability to better delineate the etiologies and ramifications of altered myocardial FA metabolism in cardiac disease. The inventors have developed radiotracers the permit assessment of multiple aspects of myocardial FA metabolism. For example, the present inventors have realized that there is no non-invasive method currently available to measure the contribution of FA from TG. They therefore have developed, in some embodiments, radiolabeled VLDL in the R1 position with an ¹⁸F-FAA (¹⁸F-FAA-VLDL). They further disclose its use in cardiac imaging and kinetic characteristics in a rat model system with and without abnormalities in myocardial FA metabolism. They furthermore disclose using the compounds to measure the production of radiolabeled metabolites in blood and myocardial tissue, and determine their whole-body biodistribution.

The inventors accordingly disclose radiolabeled tracers that are fatty acid analogs comprising a positron emitter such as ¹⁸F, or radiolabeled very low density lipoprotein triglycerides (VLDL) comprising a positron emitter such as ¹⁸F.

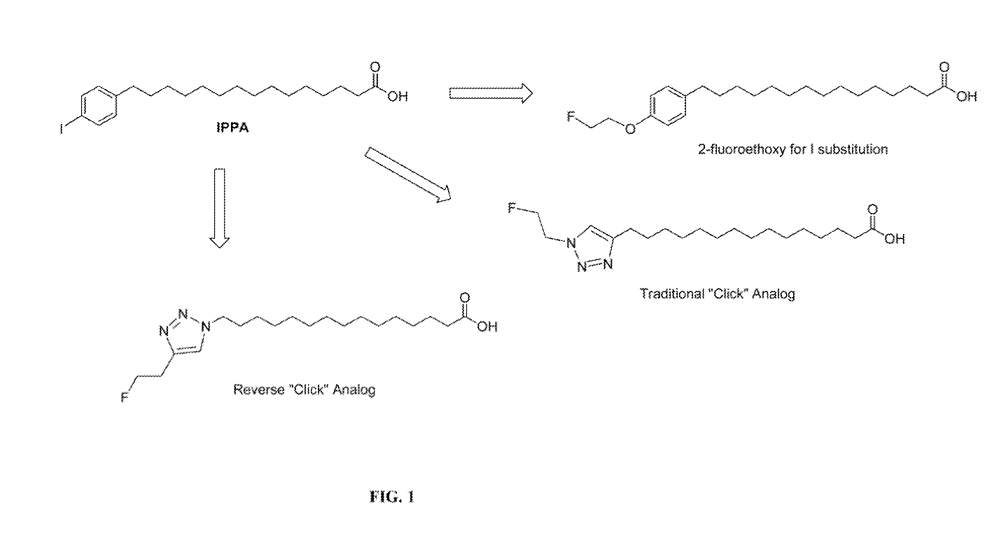

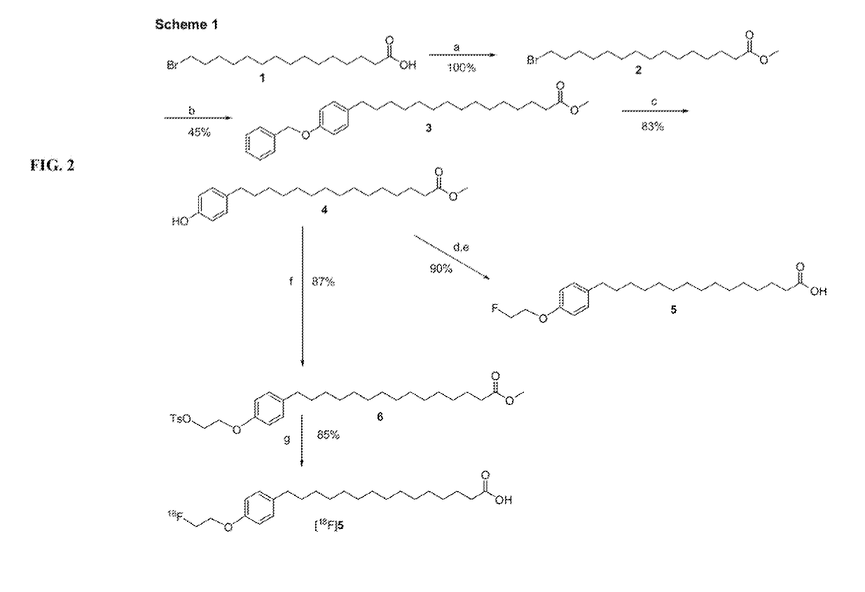

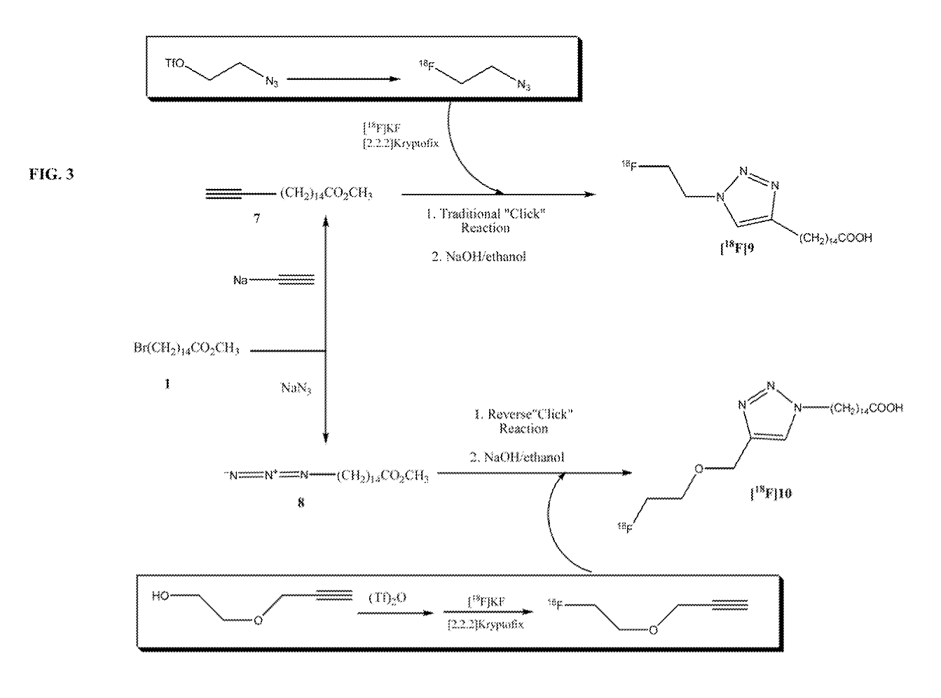

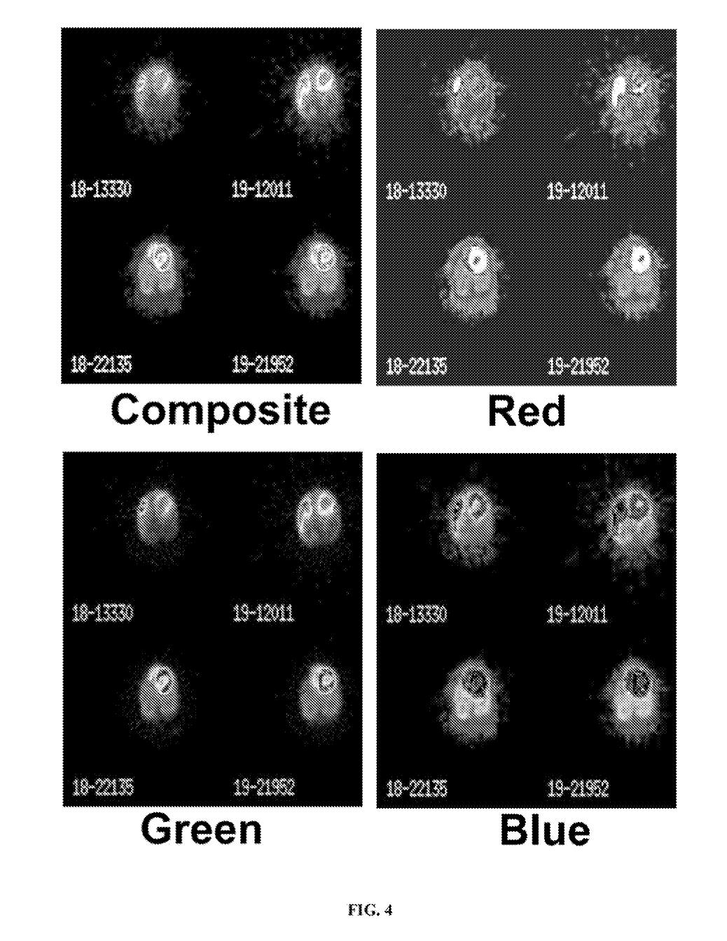

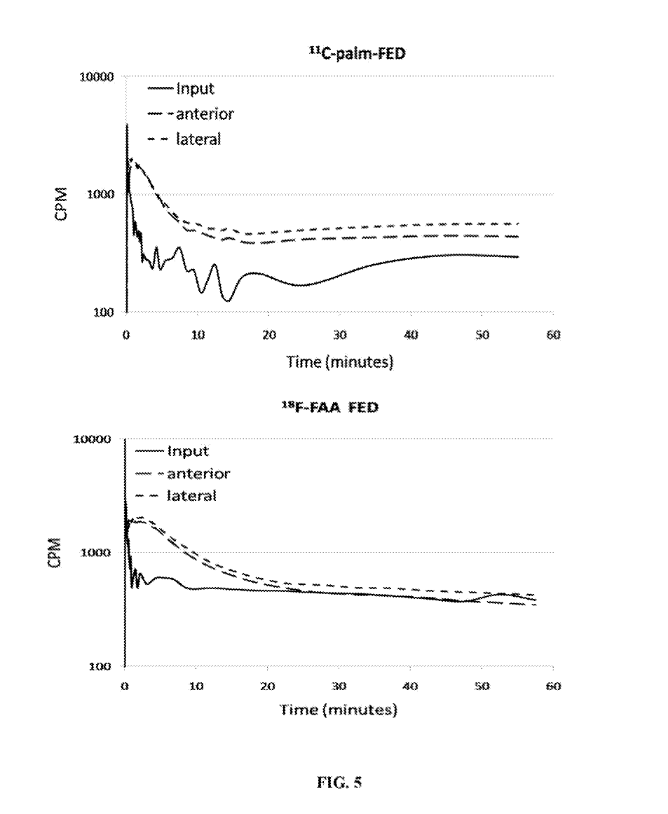

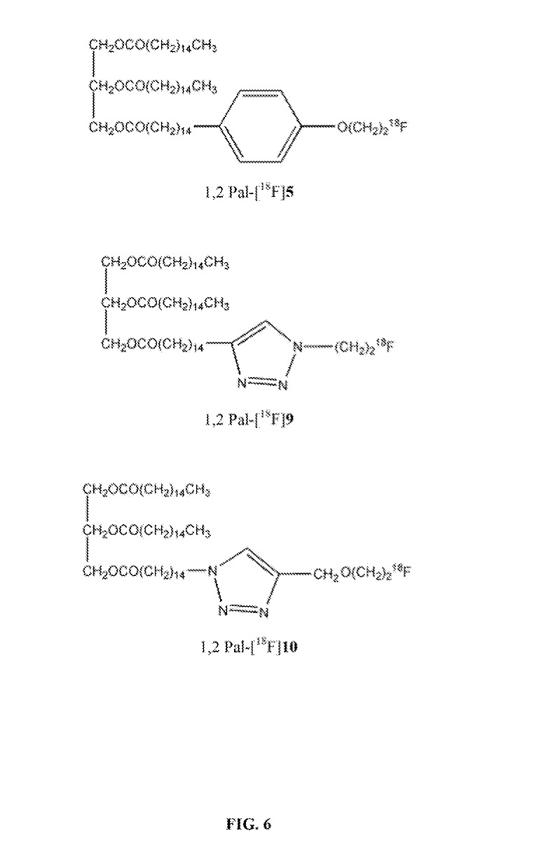

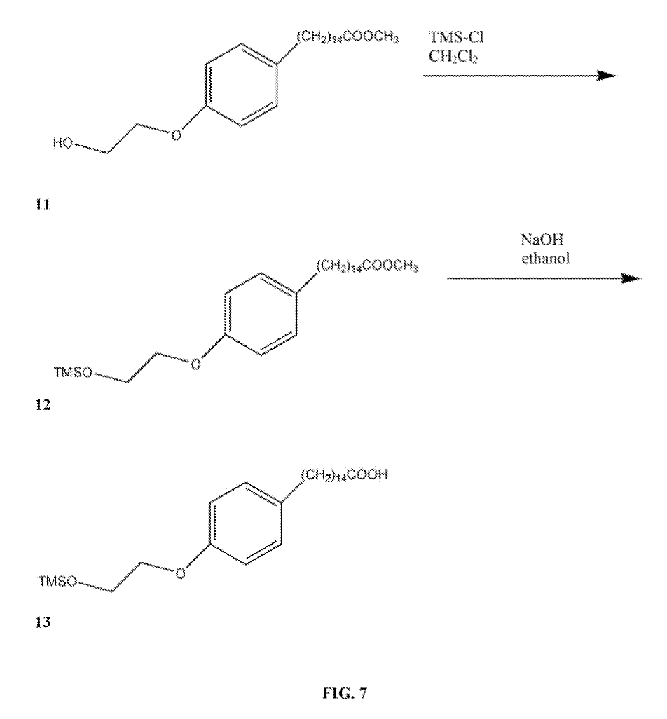

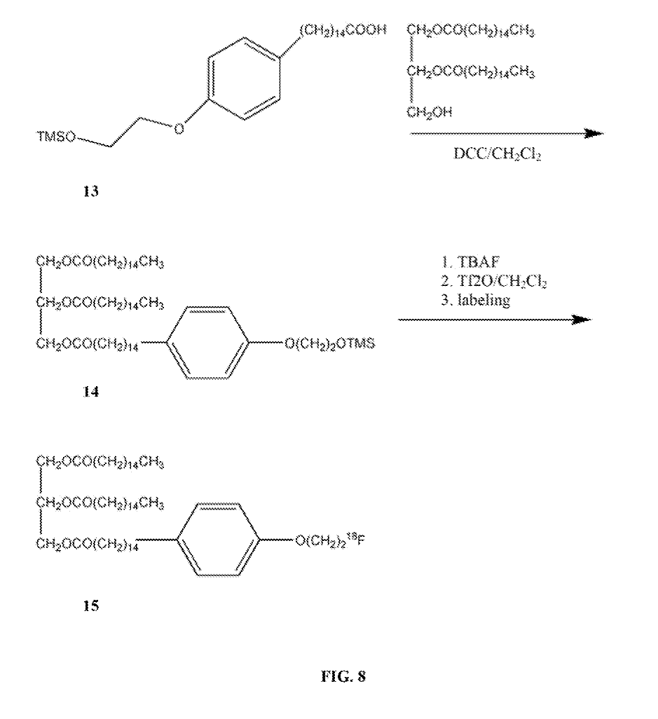

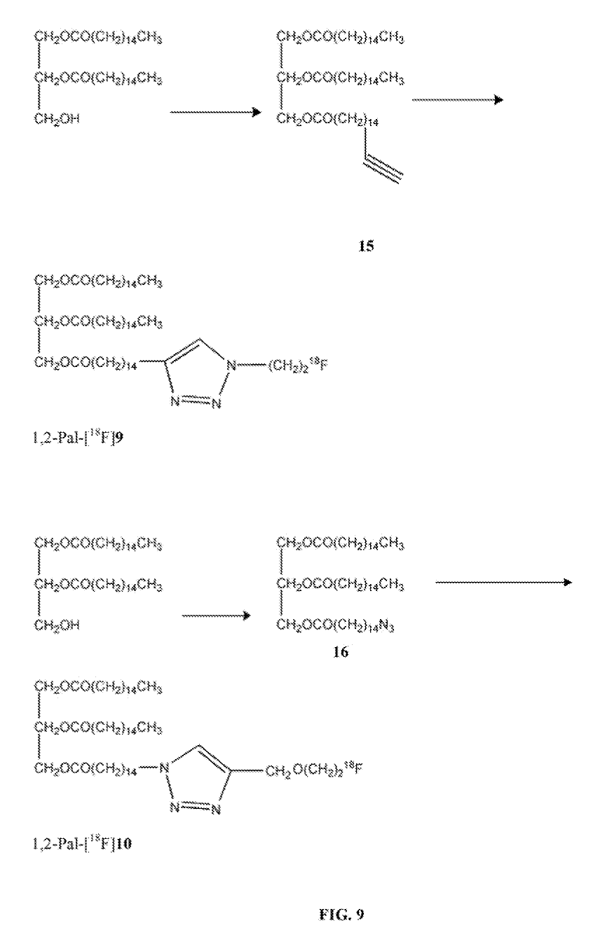

Some aspects of the present teachings involve replacing the iodo-group of IPPA with a 2-fluoroethoxy group. In some configurations, a 2-fluoroethoxy substitution can also represent an isosteric substitution for the iodo group in p-IPPA. Strategies used for synthesizing some 2-fluoroethoxy analogs of IPPA (FIG. 1) are shown in FIG. 2. In some configurations of the present teachings, a corresponding 18F-labeled analog such as,

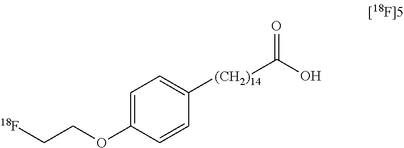

can be synthesized in high radiochemical yield (approx. 85%) from the corresponding tosyl precursor.

The methods described herein utilize laboratory techniques well known to skilled artisans, and guidance can be found in laboratory manuals and textbooks such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999; Hedrickson et al., Organic Chemistry 3rd edition, McGraw Hill, New York, 1970; Carruthers, W., and Coldham, I., Modern Methods of Organic Synthesis (4th Edition), Cambridge University Press, Cambridge, U.K., 2004; Curati, W. L., Imaging in Oncology, Cambridge University Press, Cambridge, U.K., 1998; Welch, M. J., and Redvanly, C. S., eds. Handbook of Radiopharmaceuticals: Radiochemistry and Applications, J. Wiley New York, 2003.

In the experiments described herein, all reagents were purchased from commercial suppliers and used without further purification unless otherwise stated. All reactions can be carried out under standard conditions well known to skilled artisans, such as standard air-free and moisture-free techniques under an inert argon atmosphere with dry solvents.

In various embodiments of the present teachings, methods of synthesizing ¹⁸F-labeled fatty acids based on IPPA can include provided in FIG. 1. This approach can involve replacing the iodo-group of IPPA with a 2-fluoroethoxy group. In some aspects, a 2-fluoroethoxy substitution can also be an isosteric substitution for the iodo group in p-IPPA. The synthesis of the 2-fluoroethoxy analog of IPPA as shown in FIG. 1 and the corresponding ¹⁸F-labeled analog, [¹⁸F]5 can be synthesized in high radiochemical yield (85%) from a corresponding tosyl precursor. In various aspects, ¹⁸F can be incorporated into a compound by reacting the compound with [¹⁸F]fluoride/potassium carbonate and 4,7,13,16,21,24-Hexaoxa-1,10-diazabicyclo[8.8.8]-hexacosane (Krypofix 222®, Acros Organics N.V., Fairlawn, N.J.). The reaction conditions are well known to skilled artisans. In some configurations, the reaction conditions can include using acetonitrile (MeCN) as the solvent, 110° C./5-10 min.

In other embodiments, two “click” analogs can be synthesized as outlined in FIG. 3. In these syntheses, the 4-iodophenyl ring of IPPA can be replaced with the corresponding 1,2,3-triazole moiety that is created from the “click” and “reverse click” labeling procedures. In various configurations, a click chemistry reaction can use an intermediate, such as [¹⁸F]2-fluoro-1-azidoethane to give [¹⁸F]9. In other configurations, a “reverse click” approach can include a modified click reaction, in which an ¹⁸F-radiolabel can be incorporated into an acetylene precursor, and an azido moiety for a 1,3-dipolar cycloaddition reaction can be attached to a fatty acid group, to give a compound such as, for example [¹⁸F]10.

Synthesis and Packaging of Radiolabeled Triglycerides. Certain embodiments include radiolabeled TG that can be incorporated into VLDL-TG. These embodiments can involve incorporating an ¹⁸F-FAA described above into the 1-position of a triglyceride. Examples of structures of the target TG are shown in FIG. 6. In some configurations, starting materials for the synthesis of the target compounds can be commercially available 1,2-dipalmitoyl glycerol. Conversion of the 1,2-di-palmitoylglycerol into a TG analog can be accomplished using the sequence of reactions described FIG. 7 and FIG. 8.

The present teachings include embodiments in which FAA-triglyceride analogs such as 1,2-pal-[¹⁸F]5,1,2-pal-[¹⁸F]9 and 1,2-pal-[¹⁸F]10 can be synthesized and evaluated as the enantiomeric mixtures, because the 2-position of the TG in some configurations can be a chiral center. Hence, in some configurations, a racemic mixture can be separated into (+)- and (−)-isomers using standard methods known to skilled artisans such as chiral HPLC.

### EXAMPLES

Unless results are presented in the past tense, the presentation of experiments does not imply that any protocol or experiment has, or has not, been conducted. None of the examples shall be deemed to be limiting of the scope of the disclosure.

### Example 1

### Small Animal Imaging

Animal Preparation. All animal procedures are conducted in compliance with the guidelines for the care and use of research animals established by the Animal Studies Committee of Washington University. Animal preparation is performed as described previously.²⁴⁻²⁶ Rats are housed in metabolism cages and anesthetized by inhalation of 2%-25% isoflurane administered via an induction chamber. Anesthesia can be maintained throughout the imaging session by delivering 1%-1.5% isoflurane via a custom-designed nose cone. Venous access is via the right jugular vein. Body temperature can be maintained using a circulating water blanket and a heat lamp. Heart and respiration rates can be monitored throughout the process.

PET Acquisition. The animals can be secured in a custom-designed acrylic restraining device and placed inside the field of view of the small-animal imaging PET scanner. Imaging acquisition starts Ss after a bolus injection of tracer via the right jugular catheter. The imaging protocol consists of dynamic acquisition of microPET images of ¹¹C-palmitate (5-7mCI) followed by ¹⁸F-FAA or by ¹⁸F-VLDL (5-7mCi) alone. Dynamic mage acquisition during ¹¹C-palmitate and either ¹⁸F-FAA or ¹⁸F-FAA-VLDL can be 30 and 60 min, respectively. The total time of the imaging session can be ˜3 hrs. Three whole-blood arterial samples are collected throughout the study to measure whole blood glucose (5 μL), free fatty acid (20 μL), and insulin (5 μL) levels to confirm the metabolic state of the animals,

Image Processing/Analysis. Dynamic images can be reconstructed using filtered backprojection with a 2.5 zoom on the heart and 40 frames per imaging session. The input function is reconstructed by applying the hybrid image-blood-sampling algorithm as described previously.²⁵ A myocardial region of interest is placed to generate a myocardial time-activity curve (TAC). The ¹⁸F-FAA and ¹⁸F-FAA-VLDL blood and myocardial TACs can be compared with the ¹¹C-palmitate TACs for both defiing characteristics (e.g., height and shape) as well as similar kinetics based exponential curve-fitting algorithms.

### Example 2

### Large Animal Imaging

Animal Preparation. Purpose bred ˜6-10 kg male beagle dogs are fasted, anesthetized and instrumented as reported previously^{3,4}. One femoral vein is camnulated to administer drugs. Catheters are placed in the thoracic aorta via the femoral arteries for arterial sampling and monitoring of arterial blood pressure. To obtain venous blood samples, a coronary sinus catheter can be placed via the right external jugular vein under fluoroscopic guidance as previously described.²⁸. The ECG, arterial blood pressure and heart rate can be monitored throughout the process. All measurements can be performed on the microPET Focus 220. All procedures are conducted in compliance with the Guidelines for the Care and Use of Research Animals.

Pet Imaging Protocol. Two Imaging Protocols can be Used.

Protocol 1. A transmission scan can be performed initially to correct for photon attenuation. Following the transmission scan, 5-7 mCi of ¹⁵O-water can be administered as an intravenous bolus, with the immediate initiation of dynamic data collection for 5 min. After allowing for decay of ⁵O-water, 5-7 mCi of ¹¹C-palmitate can be administered intravenously followed by a 60 min data collection. After allowing for the decay of ¹¹C-palmitate, 5-7 mCi of the FA analog can be administered followed by a 60 min data collection Concurrent with the ¹¹C-palmitate administration, a constant infusion (0.1 umol/kg/min) of ¹³C-palmitate can be started and continued until the end of the procedure to label the triglyceride pool, ¹¹C-palmitate, ¹¹CO₂, and ¹⁸F-metabolites can then be assayed by paired sampling of ACS blood. Plasma insulin and substrates can be assayed at preset intervals. Consequently, dogs can be studied under resting conditions either in the fasted state (moderate FA uptake and oxidation with low storage; n=5), during hyperinsulinemic-euglycemic clamp (low uptake and oxidation with higher fractional storage; n=5) or during the administration of dobutamine (10 μg/kg/min; high uptake and oxidation with low storage; n=5), total of 15 dogs. All interventions can be performed routinely and can show the necessary stability in the substrate environment and cardiac work to perform multi-tracer studies.^{21,22,27} Myocardial tissue can also be obtained using procedures we have reported previously.^{21,22} After an imaging protocol is completed, the chest can be opened via a left thoracotomy incision. The pericardium can be opened the heart exposed. Approximately 4-5 cm parallel incisions are made on each side of the main diagonal branch of the left anterior descending artery. The myocardium between the incisions can be raised and freeze clamped using aluminum tongs cooled in liquid N and stored at −80° C.

Protocol 2: This imaging protocol and interventions can be identical to Protocol 1 except that in place of ¹⁸F-FAA, 5-7 mCi of FTP can be administered followed by 60 min of dynamic imaging. As in Protocol 1, ¹⁵O-water and ¹¹C-palmitate can be administered with imaging. Stable isotopic measurements using ¹¹C-palmitate can be performed. Blood sampling for radiolabeled metabolites and unlabeled substrates and insulin can also be performed, Myocardial tissue can be obtained at the end of the procedure.

### Example 3

This example illustrates the feasibility of our strategy, the first 60 mins of kinetics for a 2-fluoroethox analog of IPPA were compared with those of ¹¹C-palmitate in the same animal (FIG. 4). Imaging of both radiotracers was for 60 min. Composite myocardial microPET images obtained from a fed rat studied with ¹¹C-palmitate (Top) and a novel fatty acid analog labeled with ¹⁸F-FAA (Bottom). Images are displayed on the transaxial axis and represent data acquired 20-30 mins after tracer injection. ¹⁸F-FAA images displayed excellent quality and higher tracer activity than ¹¹C-palmitate images. FIG. 4 presents individual microPET images. Top row (18-13330 and 19-12011) are ¹¹C-palmitate images and bottom row (18-22135 and 19-21952) are ¹⁸F-FAA images. Increasing signal intensity is represented green to yellow to red (highest). Relatively similar tracer kinetics was noted (FIG. 5).

### Example 4

This example illustrates Blood (input) and myocardial time-activity curves (TACs) (anterior and lateral) for ¹¹C-palmitate (top) and the ¹⁸F-FAA (Bottom). Myocardial TAC's represent average tracer activity obtained from three consecutive ROIs (FIG. 5). To visually enhance differences in myocardial kinetics, a logarithmic scale was used for the Y-axis.

Both radiotracers exhibited significant tracer uptake with rapid bi-phasic washout although some differences are noted. For instance, when compared to ¹¹C-palmitate, ¹⁸F-FAA exhibited an early plateau followed by a slower early tracer clearance (0.17±0.01 vs. 0.30±0.02, P<0.0001); and a significantly higher late clearance (0.0.0030±0.0005 vs. 0.0006±0.00013, P<0.01). Tracer clearance was still prevalent at 60 mins post-tracer injection in ¹⁸F-FAA, but not in ¹¹C-palmatate, where tracer increased in both, blood and myocardium, 25 mins post tracer injection. These data demonstrate in various embodiments the properties of these compounds and their use in methodologies to assess myocardial FA metabolism.

### Example 5

This example illustrates preparation of radiolabeled fatty acid analogs that, in some embodiments can behave like ¹¹C-palmitate in vivo, but contain a positron emitting radionuclide having a longer half-life than that of ¹¹C. Previous studies have shown that ¹²³I-lPPA displays tissue-time activity curves (TACs) similar to that of ¹¹C-palmitate. That is, both radiotracers display biphasic washout kinetics from heart, which is representative of oxidation (rapid washout phase) and storage (slow washout phase). Therefore, the strategy have developed involves the replacement of the ¹²³I radiolabel with the positron emitting radionuclide, fluorine-18. One strategy for making a ¹⁸F-labeled analog of p-IPPA can be to replace the iodine atom with an [¹⁸F]2-fluoroethoxy group ([¹⁸F]5). This strategy has been used in the synthesis of receptor-based PET radiotracers and the 2-fluoroethoxy group can be expected to be stable with respect to in vivo defluorination. As shown in Scheme 1 (FIG. 2), [¹⁸F]S has been synthesized in high yield (˜85%) and high specific activity (˜6,000 Ci/mmol at end of synthesis). In scheme 1, synthesis steps are a) trimethylsilyl diazomethane, THF hexane, 2 hr; b) 4-benzyloxyphenylboronic acid, Pd(OAc)₂, [HP(t-Bu)₂Me]BF₄, KOt-Bu, t-amyl alcohol, argon, RT, 24 hr; c) Pd/c, EtOAc, H₂, 6 psi, 5 hr; d) 1-bromo-2-fluoroethane, K₂CO₃, acetone; e) NaOH, MeOH, CHCl₂, water; f) ethane-1,2-diyl bis(4-methlylbenzenesulfonate), K₂CO₃, CH₃CN reflux 3 hr.; g) [¹⁸F]KF/K2.2.2/K₂CO₃/CH₃CN, then NaOH, wherein K2.2.2 is 4,7,13,16,21,24-Hexaoxa-1,10-diazabicyclo[8.8.8]-hexacosane (Kryptofix 222®, Acros Organics N.V., Fairlawn, N.J.). In some embodiments, the inventors disclose the synthesis of the “click” and “reverse” click analogs, as shown in FIG. 3. In some configurations, the radiochemical yield of the click and reverse click reactions can be in excess of 80% based on starting [¹⁸F]fluoride.

### Example 6

This example illustrates using the “click chemistry” procedure for labeling FIG. 3). The first strategy involves the “traditional” click chemistry reaction using the intermediate, [¹⁸F]2-fluoro-1-azidoethane to give [¹⁸F]9.²⁹ The second approach involves a “modified” click reaction, in which the ¹⁸F-radiolabel can be incorporated into an acetylene precursor. An azido moiety required for the 1,3-dipolar cycloaddition reaction can be attached to the fatty acid group, to give [¹⁸F]10.

### Example 7

This example illustrates preparation of radiolabeled TG that can be incorporated into VLDL-TG. The strategy can involve incorporating an ¹⁸F-FAA into the 1-position of a triglyceride. Exemplary structures of a target TG are shown in FIG. 6, The starting material for the synthesis of the target compounds can be commercially available 1,2-dipalmitoyl glycerol. Conversion of the 1,2-di-palmitoylglycerol into the TG analogs can be accomplished using the sequence of reactions described in FIG. 7, FIG. 8 and FIG. 9. It should be pointed out that the 2-position of the TG is a chiral center and that analogs 1,2-pal-[¹⁸F]5,1,2-pal-[¹⁸F]9 and 1,2-pal-[¹⁸F]10 can be initially synthesized and evaluated as enantiomeric mixtures. A TG can be separated into its (+)- and (−)-isomers using routine methods, such as chiral HPLC.

### Example 8

This example illustrates packaging of radiolabeled triglycerides. In some configurations, a radiolabeled TG can be “packaged” for administration ex vivo by methods known to skilled artisans, for example the methods described by Gormsen et al.²⁰ In some configurations, using aseptic techniques, blood (3-5 mL) and (20-30 mil) can be obtained from donor rats and dogs, respectively. In some configurations, very low density lipoprotein (VLDL) can be separated via ultracentrifugation (Beckham (Instruments, Palo Alto, Calif.). The VLDL supernatant can be removed using a modified Pasteur pipette, passed through a MilliporeR filter (pore size diameter 0.22 mm), and stored 4° C. for up to 1 wk. On the day of use, the ¹⁸FAA-TG can be incorporated into a VLDL complex by sonication in a water bath at 37° C. for 30 min. The resulting solution can again be passed through a 0.22 μm filter prior to bolus injection. In some aspects, representative samples can be tested to ensure apyrogenicity and sterility. In some aspects, additional control experiments can be conducted to show that such ex vivo-labeled VLDL-TG particles can be indistinguishable from native VLDL with regard to electrophoretic properties, cholesterol-to-TG ratio, apolipoprotein B-100 (apoB-100) concentrations, and mobility on size-exclusion HPLC. In some configurations, comparable procedures can be used to prepare ¹³C-VLDL, except that VLDL isolated from a larger volume of blood (i.e. 50-60 mL) can be used take up all of the tracer.

### Example 9

This example illustrates image processing and analysis. In this example, dynamic images can be reconstructed using filtered backprojection with a 2.5 zoom on the heart and 20-40 frames per imaging session. Data reconstructions (filtered resolution of images will be 10 mm) can be performed on a Silicon Graphics Computer system and transferred via Ethernet to a Sun Ultra 10 workstation for image analysis with an image-analysis software package. Myocardial images can be reformatted to orthogonal planes where regional values for tracer kinetics and perfusion and metabolism may be obtained. In addition, antero-lateral segmental values for perfusion and FA metabolism can be obtained and then averaged to obtain values per dog for the purposes of correlating with tissue measurements of triglyceride. The regional values can be used to evaluate for regional variability and bias in the parameter estimates.

### Example 10

This example illustrates measurement of regional perfusion and metabolism. Perfusion. The measurement of myocardial perfusion can be a necessary component of the compartmental model for measuring FA metabolism and to calculate the Fick measurements of substrate use. Measurements can be performed using the well-validated modeling approach of ¹⁵O-water kinetics^{23, 30-32}.

### Example 11

This example illustrates analysis and kinetic modeling strategies for F-18 radiolabeled tracers. In some embodiments, the analysis of these ¹⁸F-FAA radiotracers can follow 2 steps:

Step 1. Qualitative and semi-quantitative analysis of microPET images and kinetics in rats. Analysis strategies include 1) visualization of image quality to assess similarities/differences in myocardial tracer distribution, such as tracer distributed primarily in myocardium vs. distributed in both myocardium and blood; 2) visualization of similarities/differences in blood and myocardial TAC's and; 3) “curve striping”, where myocardial curves can be fitted to multi-exponentials to assess differences in overall similarities/differences in uptake and clearance tracer rates.

Step 2. Development and validation of compartmental models of ¹⁸F-FAA PET kinetics in a well-controlled canine model. A number of quantitative methods can be used to validate PET metabolic tracers are used during this process.^{21,22,28} First, ACS data can be used a) to identify blood and myocardial ¹⁸F metabolites, b) to quantify their contribution to myocardial metabolism of the ¹⁸F-FAA under investigation and c) to compare these ¹⁸F metabolites to others such as ¹¹C-palmitate metabolites in blood and ¹³C-palmitate metabolites in tissue and ¹³C-VLDL in blood and tissue. Secondly, based on these observations, a preliminary model is designed, implemented and tested over a wide range of metabolic and cardiac work states studied. PET blood (corrected for ¹⁸F-metabolites) and myocardial TACs can then be fitted to the model under investigation to estimate model transfer rates (kn, min-1). At this point, a number of mathematical tools can be used to assess whether the model implemented is a faithful representation of the ¹⁸F-FFA tracer kinetics. These mathematical tools include goodness-of-fit analysis to assess how well the estimated myocardial TACs matches PET TACs, and parameter sensitivity analyses to assess how well changes in tracer metabolism can be traced by model parameters. Based on these criteria, the model can be either redesigned, or accepted. If accepted, metabolic measurements as either fractions calculated from model transfer rates, or fluxes (mL/g/min) calculated from the product of metabolic fractions and myocardial blood flow, or overall metabolic rates (nmol/g/min) calculated from the product of metabolic fluxes and plasma FA levels) can be compared to the ACS measurements. Finally biologic validation of the model can be performed by comparing model derived estimates of FA metabolism with the appropriate known standards (e.g., ¹¹C-palmitate and ¹³C-VLDL).

### Example 12

This example illustrates quantification of FA uptake with FTP. Because FTP is essentially irreversibly bound in tissue, Patlak Graphical Analysis can be performed to calculate myocardial FA uptake as has been previously reported.^{17,18} The arterial input function can be corrected for presence of plasma radiolabeled metabolites.^{17,18} LC values can be calculated based on the differences in the fractional uptake of FTP compared ¹¹C-palmitate measured by the Fick method performed with ACS. Of note, compartmental modeling approaches as described above can also be explored to determine in FA uptake and oxidation can separated using this tracer.

### Example 13

Separate from PET imaging, biodistribution studies can be conducted in order to ensure optimal imaging characteristics and to obtain to the necessary information to perform dosimetry estimates for radiopharmaceuticals ultimately developed for human use. Organs of interest can be removed and the radioactivity can be counted in a gamma counter. The % I.D./organ and % I.D./g tissue can be calculated from the slope of the standard curve of counts vs nCi (i.e., counts/nCi of radioactivity) for the gamma counter. Animals will be anesthetized using isofluorane as described above. The radiotracer (50-100 μCi for fluorine-18) can be administered via tail vein injection. The animals can be euthanized by anesthesia overdose at the time points, 10, 30, 60, and 240 min post-injection of the radiotracer.

### Example 14

This example illustrates plasma analysis. In these experiments, blood samples can be collected in dry syringes and transferred into EDTA-coated vacutainers tubes, mixed well and stored in ice until ready for assay (shortest time possible) as described previously.³³ After centrifugation at 5° C. for 5 min, 3000 rpm, 0.5 mL of plasma can be transferred to a glass test tube for extraction of the labeled lipid fractions. The extracting solvent (1N HCl-n-heptane-isopropanol 1/10/40 v/v) will be added, vortexed the tubes, followed by addition of water, in-heptane and centrifugation (4000 rpm, 4 min) to separate aqueous from organic layer. Both phases can be counted for radioactivity. Samples can be analyzed for the presence of non-esterified FAs, TGs and phospholipids. An approach can comprise the use of SPE, an aminopropyl bonded phase (SPE-NH₂ column) available from a variety of sources, Bond Elut being one of these. The work can be done initially using cartridges containing 300-500 mg of the resin and positioned on a commercially available vacuum rack. Test tubes on the bottom can be changed as needed, to collect the different fractions, these selected fractions can then be evaporated and reconstituted to a small volume to analyze by 1:1 PLC as part of the validation steps, or later after validation, counted directly on a gamma-counter to quantify radioactivity.

Extraction of Different Lipid Fractions: Portions of the organic layer above can then be passed through SPE-NH₂ columns previously conditioned with heptanes. The separation of TG, free FA and phospholipids can be carried out with sequential elution with heptanes-isopropanol (1:2 v/v) (TG and other neutral lipids), 2% acetic acid in diethyl ether (free FAs) and methanol (phospholipids). Each rack of tubes can be changed in the order suggested. The solvent composition and volumes can be optimized based on sample size, amount of resin and gross radioactivity injected. The SPE-NH₂ can be used to separate the neutral components of first extract fraction into TG, cholesterol, di- and mono-glycerides. HPLC validation can be performed with a specialty column (Waters FA analysis column) using an acetonitrile-THF-water-acetic acid solvent mixture, for the free FA and phospholipids. For the HPLC analysis of TG we can test a C-18 analytical column using dichloromethane/acetonitrile or acetone/acetonitrile with a RI detector.³⁴ The HPLC column(s) can be optimized for best separation of each fraction with authentic standards. One can also explore commercially available kits for method development specifically targeting lipids such as the one offered by Zorbax Kit SB-C18/SB-CN/SB-Phenyl 5 μm 4.6×150 mm HPLC columns, to significantly lower the retention time of short-lived C-11 metabolites.

### Example 15

This example illustrates cardiac tissue analysis. These experiments can be conducted to determine the distribution of the radiolabeled FA compounds and their metabolites in cardiac tissue. Metabolite analysis can be performed by a Folch-type extraction procedure as described by Degrado et al.¹⁷ The rat hearts can be excised at the same times as the biodistribution studies described above. They can be thoroughly homogenized and sonicated (20 s) in 7 mL chloroform methanol (2:1) at 0° C. Urea (40%, 1.75 mL) and 5% sulfuric acid (1.75 mL) can be added and the mixture sonicated for an additional 20 s. After centrifugation for 10 min, aqueous, organic, and protein interphase fractions can be separated and counted. The organic phase can be further analyzed by silica-gel TLC for radiolabeled diglycerides, PA, TG, and cholesterol ester as previously described.¹⁷ Validation of the results obtained by TLC of the tissue analysis can also be done using the HPLC method previously developed.

### Example 16

This example illustrates measurement of plasma ¹³C-palmitate or ¹³C-VLDL enrichment, For measurement of 13C-palmitate or 13C-VLDL enrichment, plasma can be separated by centrifugation, heated at 60° C. for ≧15 min to destroy lipoprotein lipase activity, and stored at 80° C. until subsequent analysis. ¹³C-palmitate enrichment can be measured via gas chromatography mass spectrometry (GCMS: Agilent Technologies 5973N, Santa Clara, (CA) using the methyl ester derivative as previously described.^{35 13}C-VLDL enrichment can be determined similarly after first separating VLDL triglycerides from other lipid classes via ultracentrifugation as described by Patterson et al.³⁶

### Example 17

This example illustrates measurement of blood ¹³CO₂ enrichment and content. In these experiments, blood ¹³CO₂ enrichment can be measured by deproteinizing 0.5 mL of blood with 0.5 mL of 6 N and analyzing the headspace gas using conventional isotope ratio mass spectrometry (IRMS; Finnigan MAT Delta+ XL, Thermo Fisher Scientific, Waltham, Mass.). Blood CO₂ content can be calculated from measurement of plasma pCO2, pH, temperature, and hemoglobin concentration.³⁷ Blood ¹³CO₂ content can then calculated as the product of the ¹³CO₂ enrichment and total CO2 content.

### Example 18

This example illustrates measurement of tissue ¹³C-triglyceride and ¹³C-phospholipid enrichment and content: To determine the incorporation of ¹³C from either ¹³C-palmitate or ¹³C-VLDL into myocardial TG or phospholipid stores, frozen tissue samples can be powdered under liquid Nz, extracted using chloroform:methanol (2:1), and stored at 80° C. until subsequent analysis. A small portion of the crude lipid extract can be used to measure total trigylceride and phospholipid content using commercial available kits (L-Type triglyceride H and Phospholipids C kits, Wako Chemicals USA, Richmond, Va.). The remainder can be purified using solid phase extraction and the ¹³C;¹²C ratio of palmitate in the triglyceride and phospholipid fractions determined using gas chromatography combustion IRMS (Finnigan MAT Dela+ XL, Thermo Fisher Scientific, Waltham, Mass.), The total ¹³C palmitate content in triglycerides or phospholipids can then be derived by multiplying the tissue content (in μmol/g) by the corresponding enrichment (tracer:tracee ratio). These data can then be used along with the precursor (i.e., ¹³C-palmitate or ¹³C-VLDL) enrichment and the duration of tracer infusion to calculate the actual rate of triglyceride or phospholipid synthesis (in μmol/g/min).

### Example 19

This example illustrates Measurement of plasma substrates and insulin. In these experiments, plasma glucose and lactate levels can be assayed enzymatically using a 2300 STAT Plus Analyzer (YSI Life Sciences, Yellow Springs, Ohio). Plasma free FA levels can be measured using an enzymatic colorimetric method (Wako NEFA C kit, Wako Chemicals USA, Richmond, Va.). Plasma insulin can be measured by radioimmunoassay (Linco Research Co., St. Charles, Mo.).

### Example 20

This example illustrates imaging protocols. Protocol 1: In these experiments, all imaging studies can be performed on the microFocus 220. The electrocardiogram, arterial blood pressure, and blood gas values can be monitored continuously. A transmission scan can be performed initially to correct for photon attenuation. Following the transmission scan, 5-7 mCi of ¹⁵O-water can be administered as an intravenous bolus, with the immediate initiation of dynamic data collection for 5 min. After allowing for decay of ¹⁵O-water, 5-7 mCi of ¹¹C-palmitate can be administered intravenously followed by a 60 min data collection. After allowing for the decay of ¹¹C-palmitate, 5-7 mCi of the FA analog can be administered followed by a 60 min data collection. Concurrent with the ¹¹C-palmitate administration, a constant infusion (0.1 umol/kg/min) of ¹³C-palmitate can be started and continued until the end of the study to label, the triglyceride pool. ¹¹C-palmitate, ¹¹CO2, and ¹⁸F-metabolites can be the assayed by paired sampling of ACS blood. Plasma insulin and substrates can be assayed at preset intervals. The goal here can be to achieve a wide range in both the rate of myocardial FA utilization and the metabolic fate of extracted FA with respect to storage in primarily TG, (and to a lesser extent phospholipids and neutral lipids) and β-oxidation. Consequently, dogs can be studied under resting conditions either in the fasted state (moderate FA uptake and oxidation with low storage; n=5), during hyperinsulinemic-euglycemic clamp (low uptake and oxidation with higher fractional storage; n=5) or during the administration of dobutamine (10 μg/kg/min; high uptake and oxidation with low storage; n=5), total of 15 dogs. Next myocardial tissue can be obtained using procedures we have reported previously.^{21,22} After an imaging protocol is completed, the chest can be opened via a left thoracotomy incision. The pericardium can be opened the heart exposed. Approximately 4-5 cm parallel incisions can be made on each side of the main diagonal branch of the left anterior descending artery. The myocardium between the incisions can be raised and freeze clamped using aluminum tongs cooled in liquid N₂ and stored at −80° C. This procedure can permit continued perfusion of tissue prior to freezing to ensure stable glycogen stores. Animals can be euthanized with an overdose of sodium thiopental (at least 60 mg/kg) followed 1-2 minutes later with 60 mL of saturated KC will be given via the left atrial or left ventricular catheter.

Protocol 2: This imaging protocol and interventions are identical to Protocol 1 except that in place of ¹⁸F-FAA 5-7 mCi of FTP can be administered followed by 60 min of dynamic imaging. As in Protocol 1, ¹⁵O-water and ¹¹C-palmitate can be administered with imaging. Stable isotopic measurements using ¹³C-palmitate can be performed. Blood sampling for radiolabeled metabolites and unlabeled substrates and insulin can also be performed. Myocardial tissue can be obtained at the end of a procedure.

### Example 21

This example illustrates data analysis.

In the development of our ¹¹C-glucose and L-3-¹¹C-lactate models, sample sizes of 20 and 23 dogs were used.^{22,28} For Protocol 1, univariate analyses can be performed to determine if Fick-derived values for myocardial uptake and measurements of its myocardial clearance (as a measure of oxidation) of the ¹⁸F-FAA track with changes in substrate and hormonal availability. Differences between grouped data can be compared using analysis of variance with the post-hoc Scheffé test. If so, these values can then be correlated with similar measurements of ¹¹C-palmitate uptake and ¹¹CO₂ production. Comparison of uptake values can also help determine if a LC can be included the determination of myocardial FA uptake using the ¹⁸F-FAA. Next, employing the general modeling paradigm described above, a compartmental model based on the myocardial kinetics of the ¹⁸F-FAA can be developed. Univariate analyses of the various modeling parameters such as FA uptake, oxidation and storage can be performed to determine if track with changes in substrate and hormonal availability. Tomographic estimates of FA uptake, oxidation and storage using the ¹⁸F-FAA (as the dependent variable) can be compared with PET derived values using ¹¹C-palmitate (as the independent variable) using standard regression analysis. Furthermore, measured rates of FA storage can be correlated with the directly-determined rate of incorporation of ¹³C-palmitate into TO and phospholipid.

For Protocol 2, univariate analyses can be performed to determine if Patlak-derived FTP rates of FA uptake track with changes in substrate and hormonal availability.

Tomographic estimates of uptake using FTP (as the dependent variable) can be compared with Fick-derived measurements of uptake using ¹¹C-palmitate (as the independent variable) using standard regression analysis. In addition, LC values can be calculated for the 3 interventions to determine its stability under these conditions. A similar comparison can be performed with FTP derived measurements of FA uptake with ¹¹CO₂ production to determine if the uptake parameter measured with FTP is more indicative of oxidation. Moreover, correlations between the PET-derived values for FA uptake (with FTP) and FA uptake (with the ¹⁸F-FAA; from Protocol 1) with the Fick-derived values using ¹¹C-palmitate can be compared to determine if the ¹⁸F-FAA provides more accurate measurements of FA uptake under the conditions studied. As mentioned previously, potential compartmental modeling approaches with FTP can also be explored. Values for FA uptake, oxidation and although unlikely, storage, can be compared with Fick-derived values using ¹¹C-palmitate and tissue measurements of ¹³C-palmitate, respectively.

### REFERENCES

1. Chatham J C, Gao Z P, Forder J R. Impact of 1 wk of diabetes on the regulation of myocardial carbohydrate and fatty acid oxidation. Am J Physiol. August 1999; 277(2 Pt 1):E342-351.
2. Herrero P, Peterson L R, McGill J B, Matthew S, Lesniak D, Dence C, Gropler I. Increased myocardial fatty acid metabolism in patients with type 1 diabetes mellitus. J Am Coll Cardiol. Feb. 7, 2006; 47(3):598-604.
3. Augustus A S, Kako Y, Yagyu H, Goldberg I J. Routes of FA delivery to cardiac muscle: modulation of lipoprotein lipolysis alters uptake of TO-derived FA. Am J Physiol Endocrinol Metab. February 2003; 284(2):E331-339.
4. Noh H L, Yamashita H, Goldberg I J. Cardiac metabolism and mechanics are altered by genetic loss of lipoprotein triglyceride lipolysis. Cardiovasc Drugs Ther. December 2006; 20(6):441-444.
5. Chatham J C, Des Rosiers C, Forder J R. Evidence of separate pathways for lactate uptake and release by the perfitsed rat heart. Am J Physiol Endocrinol Metab. October 2001; 281(4): E794-802.
6. Lewandowski E D. Cardiac carbon 13 magnetic resonance spectroscopy: on the horizon or over the rainbow? J Nucl Cardiol. July-August 2002; 9(4):419-428.
7. Lewandowski E D, Kudej R K, White L T, O'Donnell J M, Vatner S F. Mitochondrial preference for short chain fatty acid oxidation during coronary artery constriction. Circulation. Jan. 22, 2002; 105(3):367-372.
8. DeGrado T R, Holden J E, Ng C K, Raffel D M, Gatley S J. Quantitative analysis of myocardial kinetics of 15-p-[iodine-125]iodophenylpentadecanoic acid. J Nucl Med. July 1989; 30(7):1211-1218.
9. Dormehl I C, Hugo N, Rossouw D, White A, Feinendegen L E. Planar myocardial imaging in the baboon model with iodine-123-15-(iodophenyl)pentadecanoic acid (IPPA) and iodine-123-15-(P-iodophenyl)-3-R,S-methylpentadecanoic acid (BMIPP), using time-activity curves for evaluation of metabolism. Nucl Med Biol. October 1995; 22(7):837-847.
10. Eckelmarn W C, Babich J W. Synthesis and validation of fatty acid analogs radiolabeled by nonisotopic substitution. J Nucl Cardiol May-June 2007; 14(3 Suppl):S100-109,
11. Sloof G W, Visser F C, Eenige van M J, Comans E F, Teerlink T, Herscheid J D, Van der Vusse G J, Knapp F F, Jr. Comparison of uptake, oxidation and lipid distribution of 17-iodoheptadecanoic acid, 15-(p-iodophenyl)pentadecanoic acid and 15-(p-iodophenyl)-3,3-dimethylpentadecanoic acid in normal canine myocardium. J Nucl Med. April 1993; 34(4):649-657.
12. Ambrose K R., Owen B A, Goodman M M, Knapp F F, Jr. Evaluation of the metabolism in rat hearts of two new radioiodinated 3-methyl-branched fatty acid myocardial imaging agents. Eur J Nucl Med. 1987:12(10):486-491.
13. Goodman M M, Kirsch G, Knapp F F, Jr. Synthesis and evaluation of radioiodinated terminal p-iodophenyl-substituted alpha- and beta-methyl-branched fatty acids. J Med Chem. March 1984; 27(3):390-397.
14. Knapp F F, Jr., Kropp J. Iodine-123-labelled fatty acids for myocardial single-photon emission tomography: current status and future perspectives. Eur J Nucl Med. April 1995; 22(4):361-38.
15. DeGrado T R. Synthesis of 14(R,S)-[18F]fluoro-6-thia-heptadecanoic acid (FTHA). J Label Comp Radiopharm. 1991; 29:989-995.
16. DeGrado T R, Coenen H H, Stocklin G. 14(R,S)-[18F]fluoro-6-thia-heptadecanoic acid (FTHA): evaluation in mouse of a new probe of myocardial utilization of long chain fatty acids. J Nucl Med. October 1991; 32(10): 1888-1896.
17. DeGrado T R, Wang S, Holden J E, Nickles R J, Taylor M, Stone C K. Synthesis and preliminary evaluation of (18)F-labeled 4-thia palmitate as a PET tracer of myocardial fatty acid oxidation. Nucl Med Biol. April 2000; 27(3):221-231.
18. DeGrado T R, Kitapci M T, Wang S, Ying J, Lopaschuk G D. Validation of 18F-fluoro-4-thia-palmitate as a PET probe for myocardial fatty acid oxidation: effects of hypoxia and composition of exogenous fatty acids. J Nucl Med. January 2006; 47(1):173-18.
19. Shoup T M, Elmaleh D R, Bonab A A, Fischman A J. Evaluation of trans-9-18F-fluoro-3,4-Methyleneheptadecanoic acid as a PET tracer for myocardial fatty acid imaging. J Nucl Med. February 2005; 46(2):297-304.
20. Gormsen L C, Jensen M D, Nielsen S. Measuring VLDL-triglyceride turnover in humans using ex vivo-prepared VLDL tracer. J Lipid Res. January 2006; 47(1):99-106.
21. Herrero P, Kisrieva-Ware Z, Dence C S. Patterson B, Coggan A R, Han D H, Ishii Y, Eisenbeis P, Gropler R J. PET measurements of myocardial glucose metabolism with 1-11C-glucose and kinetic modeling. J Nucl Med. June 2007; 48(6):955-964.
22. Herrero P, Dence C S, Coggan A R, Kisrieva-Ware Z, Eisenbeis P, Gropler R J. L-3-11C-lactate as a PET tracer of myocardial lactate metabolism: a feasibility study. J Nucl Med. December 2007; 48(12):2046-2055.
23. Herrero P, Kim J, Sharp T L, Engelbach J A, Lewis J S, Gropler R J, Welch M J. Assessment of myocardial blood flow using 15O-water and 1-11C-acetate in rats with small-animal PET. J Nucl Med. March 2006; 47(3):477-485.
24. Sharp T L, Dence C S, Engelbach J A, Herrero P, Gropler R J, Welch M J. Techniques necessary for multiple tracer quantitative small-animal imaging studies. Nucl Med Biol. November 2005; 32(8):875-884.
25. Shoghi K I, Gropler R J, Sharp T, Herrero P, Fettig N, Su Y, Mitra M S, Kovacs A, Finck B N, Welch M J. Time course of alterations in myocardial glucose utilization in the Zucker diabetic fatty rat with correlation to gene expression of glucose transporters: a small-animal PET investigation. J Nucl Med. August 2008; 49(8): 1320-1327.
26. Welch M J. Lewis J S, Kim J, Sharp T L, Dence C S, Gropler R J, Herrero P. Assessment of myocardial metabolism in diabetic rats using small-animal PET: a feasibility study. J Nucl Med. April 2006; 47(4):689-697.
27. Herrero P, Sharp T L, Dence C, Haraden B M, Gropler R J. Comparison of 1-(11)C-glucose and (18)F-FDG for quantifying myocardial glucose use with PET. J Nucl Med. November 2002; 43(11):1530-1541.
28. Herrero P, Weinheimer C J, Dence C, Oellerich W F, Gropler R J. Quantification of myocardial glucose utilization by PET and 1-carbon-11-glucose. J Nucl Cardiol. January-February 2002; 9(1):5-14.
29. Glaser M, Arstad E. “Click labeling” with 2-[18f]fluoroethylazide for positron emission tomography. Bioconjug Chem. May-June 2007; 18(3):989-993.
30. Bergmann S R, Fox K A, Rand A L, McElvany K D, Welch M J, Markham J, Sobel B E. Quantification of regional myocardial blood flow in vivo with H215O. Circulation. October 1984; 70(4):724-733.
31. Peterson L R, Eyster D, Davila-Roman V G, Stephens A L, Schechtman K B, Herrero P, Gropler R J. Short-term oral estrogen replacement therapy does not augment endothelium-independent myocardial perfusion in postmenopausal women. Am Heart J. October 2001; 142(4):641-647.
32. Srinivasan M, Herrero P, McGill J B, Bennik J, Heere B, Lesniak D, Davila-Roman V G, Gropler R J. The effects of plasma insulin and glucose on myocardial blood flow in patients with type 1 diabetes mellitus. J. Am. Coll Cardiol. Jul. 5, 2005; 46(1):42-48.
33. Guiducci L, Jarvisalo M, Kiss J, Nagren K, Viijanen A, Naum A G, Gastaldelli A, Savunen T, Knuuti J, Salvadori P A, Ferrannini E, Nuutila P, Iozzo P. [11C]palmitate kinetics across the splanchnic bed in arterial, portal and hepatic venous plasma during fasting and euglycemic hyperinsulinemia. Nucl Med Biol. May 2006; 33(4):521-528.
34. Moreda W, Perez-Camino M C. Cert A. Grasa y Aceites. 2003; 54: 175-179.
35. Coggan A R, Kisrieva-Ware Z, Deuce C S, Eisenbeis P, Gropler R J, Herrero P. Measurement of myocardial fatty acid esterification using [1-11C]palmitate and PET: comparison with direct measurements of myocardial triglyceride synthesis. J Nucl Med. under review.
36. Patterson B W, Mittendorfer B, Elias N, Satyanarayana R, Klein S. Use of stable isotopically labeled tracers to measure very low density lipoprotein-triglyceride turnover. J Lipid Res. February 2002; 43(2):223-233,
37. Douglas A R, Jones N L, Reed J W. Calculation of whole blood CO2 content. J Appl Physiol. July 1988; 65(1):473-477.

All cited references are hereby incorporated by reference, each in its entirety as if fully set forth herein. Citation of a reference herein shall not be construed as an admission that such is prior art relevant to patentability of the present invention.

What is claimed is:

1. A fatty acid analog or salt thereof of structure 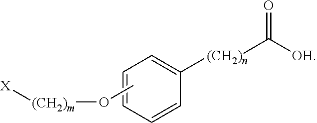
wherein n is an integer from 10 to 24, m is an integer from 1 to 10, and X is a halogen.

2. A fatty acid analog or salt thereof in accordance with claim 1, wherein the fatty acid analog is

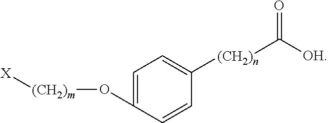

3. A fatty acid analog or salt thereof in accordance with claim 2, wherein X is a fluorine.

4-5. (canceled)

6. A fatty acid analog or salt thereof in accordance with claim 2, wherein X is an ¹⁸F.

7. A fatty acid analog or salt thereof in accordance with claim 2, wherein m=2.

8. A fatty acid analog or salt thereof in accordance with claim 2, wherein n=14.

9. A fatty acid analog or salt thereof in accordance with claim 1, wherein n=14, m=2 and X is ¹⁸F.

10-20. (canceled)

21. An ¹⁸F-fatty acid analog-very low density lipoprotein triglyceride (¹⁸F-FAA-VLDL) of structure 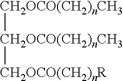
or a salt thereof, wherein R is 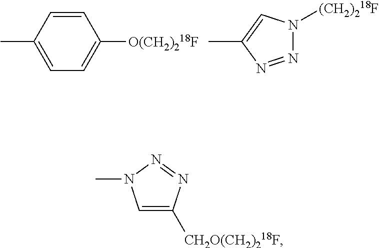
and wherein n is an integer from 10 to 24.

22. An ¹⁸F-FAA-VLDL or salt thereof in accordance with claim 21, wherein n=14.

23-30. (canceled)

31. A method of synthesizing 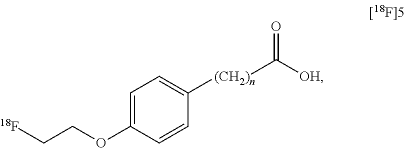
comprising contacting 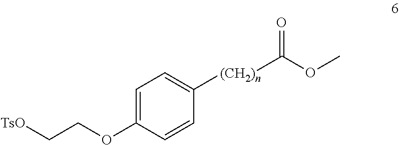
with [¹⁸F]KF/K2.2.2/K₂CO₃/CH₃CN, then NaOH,
wherein n is an integer from 10 to 24.

32. A method in accordance with claim 31, wherein n=14.

33. A method in accordance with claim 31, further comprising:
a) contacting Br—(CH₂)ₙ—COOH 1 with trimethylsilyl diazomethane and THF to yield Br—(CH₂)ₙ—COOCH₃ 2;
b) contacting the Br—(CH₂)ₙ—COOCH₃ 2 with 4-benzyloxyphenylboronic acid, Pd(OAc)₂, [Hp(t-Bu)₂Me]BF₄, KOt-Bu and t-amyl alcohol to yield 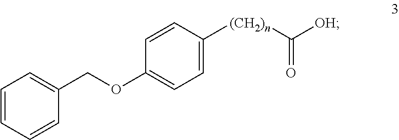
c) contacting the 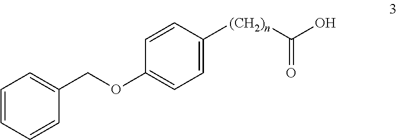
with of Pd/c and EtOAc to yield 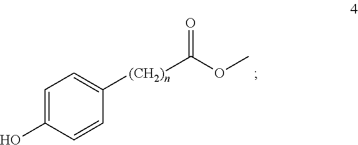
d) contacting the 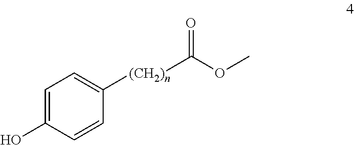
with ethane-1-2-diyl bis(4-methylbenzenesulfonate), K₂CO₃ and CH₃CN to yield

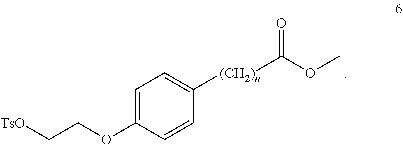

34-45. (canceled)

46. A method of synthesizing 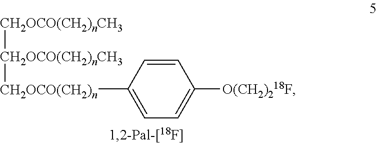
comprising:
contacting 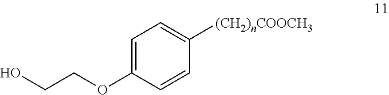
with TMS-Cl and CH₂Cl₂ to form 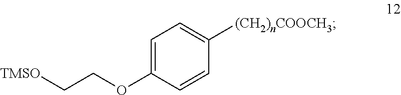
contacting the 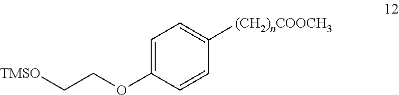
with NaOH and ethanol to form 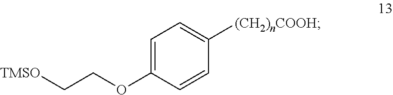
contacting the 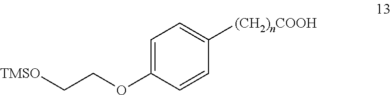
with 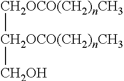
and DCC/CH₂Cl₂ to form 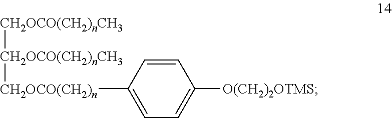
and
contacting the 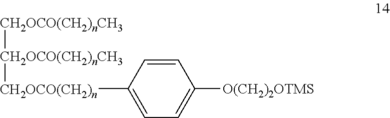
with TBAF, then Tf₂O/CH₂Cl₂, then labeling, wherein n is an integer from 10 to 24.

47. A method in accordance with claim 46, wherein n=14.

48-51. (canceled)

52. A method of determining fatty acid distribution in an mammal, comprising:
administering to the mammal a radiolabeled fatty acid analog or salt thereof 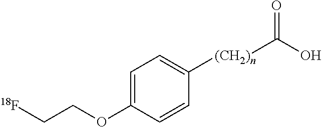
and
subjecting the mammal to positron emission tomography (PET) scanning, wherein n is an integer from 10 to 24.

53. (canceled)

54. A method in accordance with claim 52, wherein n=14.

55. A method in accordance with claim 52, further comprising subjecting image data to analysis by an algorithm in a digital computer.

56. A method in accordance claim 52, further comprising displaying the fatty acid distribution in an image on a computer display.

57. A method of imaging distribution of fatty acid triglycerides in a mammal, comprising:
administering to the mammal a radiolabeled fatty acid triglyceride analog or salt thereof 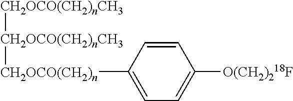
and
subjecting the mammal to positron emission tomography (PET) scanning, wherein n is an integer from 10 to 24.

58. A method in accordance claim 57, wherein n=14.

59. A method in accordance claim 57, further comprising:
subjecting image data to analysis by an algorithm in a digital computer.

60. A method in accordance with claim 57, further comprising displaying fatty acid triglyceride distribution on a computer display.
